(19) United States
(12) Patent Application Publication
(10) Pub. No.: US 2015/0297188 A1
(43) Pub. Date: Oct. 22, 2015

(54) SYSTEMS AND METHODS FOR ESTIMATING ACOUSTIC ATTENTUATION IN A TISSUE

(71) Applicant: The Trustees of Columbia University in the City of New York, New York, NY (US)

(72) Inventors: ELISA E. KONOFAGOU, New York, NY (US); Jiangang Chen, He Bei Province (CN); Gary Yi Hou, Campbell, CA (US)

(73) Assignee: The Trustees of Columbia University in the City of New York, New York, NY (US)

(21) Appl. No.: 14/695,674

(22) Filed: Apr. 24, 2015

Related U.S. Application Data
(63) Continuation-in-part of application No. PCT/US14/11631, filed on Jan. 15, 2014.
(60) Provisional application No. 61/753,706, filed on Jan. 17, 2013.
(60) Provisional application No. 61/983,733, filed on Apr. 24, 2014.

(51) Int. Cl. A61B 8/08; A61B 8/00; A61B 8/14

(52) U.S. Cl. CPC A61B 8/5223; A61B 8/14; A61B 8/08; A61B 8/4477

(57) ABSTRACT

Systems and techniques for estimating acoustic attenuation in a tissue from time-varying radiation force information generated through the application of acoustic energy to the tissue from at least first and second focal depths are provided. An exemplary technique includes acquiring first signals representing oscillatory motion of the tissue in response to the radiation force proximate the first focal depth, and acquiring second signals representing oscillatory motion of the tissue in response to the radiation force proximate the second focal depth. The technique further includes estimating the oscillatory motion of the tissue from each of the first and second signals, and estimating the acoustic attenuation in the tissue from the estimated oscillatory motion of the tissue from the first and second signals.

### CROSS REFERENCE TO RELATED APPLICATIONS

This application is a continuation-in-part of International Patent Application No. PCT/US2014/011631, filed Jan. 15, 2014, which claims priority to U.S. Provisional Application No. 61/753,706, filed Jan. 17, 2013, each of which is incorporated by reference herein in its entirety. This application also claims priority to U.S. Provisional Application No. 61/983,733, filed Apr. 24, 2014, which is incorporated by reference herein in its entirety.

### STATEMENT REGARDING FEDERALLY-SPONSORED RESEARCH

This invention was made with government support from the National Institutes of Health under Grant No. R01EB014496. The government has certain rights in the invention.

### BACKGROUND

Acoustic attenuation generally refers to the reduction in acoustic pressure amplitude during propagation within a medium. The ability to accurately estimate attenuation can be useful in therapeutic ultrasound, where the acoustic intensity within the region of interest (ROI) can be estimated. This can allow for improved tracking of the induced temperature rise during tissue ablation; ultrasound imaging, where precise time gain compensation can be obtained to improve the image quality; and ultrasonic tissue characterization, which can allow for microscopic examination of the scatterer size and backscatter coefficient as well as in situ temperature monitoring. In the field of acoustic radiation force imaging, attenuation can be a factor for quantifying the generated radiation force. Furthermore, attenuation can be related to tissue pathology. For example, attenuation can be varied by a factor of up to 35% between normal and alcoholic livers in human subjects, which can provide an indicator for alcoholic liver disease. In addition, attenuation can correlate with pathologic fat and fibrosis in livers. Tissue attenuation can also change during lesion formation using HIFU (high intensity focused ultrasound).

One technique for estimating acoustic attenuation is the broadband substitution method. Other techniques can include centroid and multi-narrowband techniques, which can analyze backscattered ultrasound signals in B-mode images. Applications of such techniques can include estimating the differential attenuation of HIFU-induced lesions. Although certain techniques can be suitable for estimating tissue attenuation, the application of such techniques in clinical practice can be challenging, due at least part to the diffraction effect from the finite aperture of the transducer, which can introduce undesired spectral disturbance to the acoustic wave. Influence of overlying tissues, for example the abdominal wall structure, can distort the acoustic wave in spectrum due at least in part to phase aberration effects. Likewise, effects from scatterers,can also influence the spectrum of the backscattered signal.

Certain acoustic radiation force techniques can be utilized for attenuation measurements. For example, the reduction in radiation force resulting from the insertion of a tissue sample between a transducer and a reflector can be measured for attenuation estimation. Furthermore, an attenuation estimation approach using linear array transducers can be utilized to generate a radiation force. The induced displacement can be monitored after the application of the radiation force. The ultrasound focus can be electronically shifted away from the transducer surface while keeping the f-number of the transducer constant, and the attenuation can be calculated at the focal depth, which can be where the radiation force reaches a maximum. Such techniques can be applied using conventional diagnostic scanners without additional hardware.

Harmonic Motion Imaging (HMI) is another example of a radiation-force-based technique. However, HMI can include monitoring the displacement in synchronization with the application of radiation force, which can provide tissue properties that certain other techniques cannot. HMI can also be used to monitor thermal ablation based on the displacement variations due to changes in tissue stiffness during ablation, and to evaluate changes in the tissue viscoelasticity parameters. Improving the ability of HMI to quantify the Young's modulus of soft tissues can be beneficial in implementing clinically translatable mechanical testing systems and techniques for in vivo application. However, the radiation force exerted within the excitation region is not necessarily known.

### SUMMARY

Techniques for estimating acoustic attenuation in a tissue are disclosed herein.

In one embodiment of the disclosed subject matter, methods are provided for estimating acoustic attenuation in a tissue from time-varying radiation force information generated through the application of acoustic energy to the tissue from at least first and second focal depths. An example method includes acquiring first signals representing oscillatory motion of the tissue in response to the radiation force proximate the first focal depth, and acquiring second signals representing oscillatory motion of the tissue in response to the radiation force proximate the second focal depth. The method further includes estimating the oscillatory motion of the tissue from each of the first and second signals, and estimating the acoustic attenuation in the tissue from the estimated oscillatory motion of the tissue from the first and second signals.

In some embodiments, the method can include applying the acoustic energy by pulsing a focused ultrasound transducer at a modulation frequency. Acquiring each of the first and signals can include pulsing an imaging transducer configured as a pulser/receiver to acquire radio frequency signals at a pulse repetition frequency.

In some embodiments, estimating the oscillatory motion of the tissue from each of the first and second signals can include applying 1D normalized cross correlation to the acquired radio frequency signals. Additionally or alternatively, estimating the acoustic attenuation can include linearly correlating the estimated oscillatory motion from each of the first and second signals.

In some embodiments, the method can include estimating the acoustic attenuation at a first portion of the tissue, estimating the acoustic attenuation at a second portion of the tissue lateral from the first portion, and determining a displacement map of the tissue using the estimated acoustic attenuation of the first portion and the estimated acoustic attenuation of the second portion.

In another aspect of the disclosed subject matter, systems for estimating acoustic attenuation in a tissue are provided, and generally include an ultrasound transducer an imaging transducer, one or more memories and a processor. In an example, the ultrasound transducer is configured to apply acoustic energy to the tissue a first focal depth and a second focal depth to generate a time-varying radiation force proximate the first focal depth and the second focal depth. The imaging transducer is configured to be optically coupled to the tissue and acquire first signals representing oscillatory motion of the tissue in response to the radiation force proximate the first focal depth and second signals representing oscillatory motion of the tissue in response to the radiation force proximate the second focal depth. The one or more processors are coupled to the one or more memories and the imaging transducer and configured to estimate the oscillatory motion of the tissue from each of the first and second signals; and estimate the acoustic attenuation in the tissue from the estimated oscillatory motion of the tissue from the first and second signals.

In some embodiments, the one or more processors can be coupled to the ultrasound transducer and can be further configured to pulse the ultrasound transducer at a modulation frequency. The imaging transducer can be configured as a pulser/receiver, and in some embodiments, the one or more processors can be further configured to pulse the imaging transducer at a pulse repetition frequency to acquire radio frequency signals corresponding to each of the first and second signals.

In some embodiments, the one or more processors can be further configured to estimate the oscillatory motion of the tissue from each of the first and second signals by applying 1D normalized cross correlation to the acquired radio frequency signals. Estimating the acoustic attenuation can include linearly correlating the estimated oscillatory motion from each of the first and second signals.

In some embodiments, the system can include a positioning apparatus coupled to the ultrasound transducer and logically coupled to the one or more processors. The positioning apparatus can be to move the ultrasound transducer to aim the ultrasound transducer at the first focal depth and the second focal depth in response to the one or more processors. The positioning apparatus can be further configured to aim the ultrasound transducer to a first portion of the tissue and a second portion of the tissue lateral from the first portion in response to the one or more processors. As such, the one or more processors can be further configured to estimate the acoustic attenuation at each of the first portion and second portion of the tissue, and determine a displacement map of the tissue using the estimated acoustic attenuation of the first portion and the estimated acoustic attenuation of the second portion. The imaging transducer can be coupled to and coaxially aligned with the ultrasound transducer.

### BRIEF DESCRIPTION OF THE DRAWINGS

The patent or application file contains at least one drawing executed in color. Copies of this patent or patent application publication with color drawings will be provided by the Office upon request and payment of the necessary fee.

The accompanying drawings, which are incorporated and constitute part of this disclosure, illustrate some embodiments of the disclosed subject matter.

FIG. 1 is a diagram illustrating an exemplary system for estimating acoustic attenuation according to the disclosed subject matter.

FIGS. 2(a)-2(b) are diagrams illustrating attenuation measurements at exemplary focal locations in a tissue.

FIGS. 3(a)-3(b) are images of exemplary HIFU lesions in in vitro canine livers.

FIG. 4 is a diagram illustrating exemplary harmonic variation in local displacement.

FIGS. 5(a)-5(j) are diagrams illustrating HMI displacement compared to acoustic intensity.

FIGS. 6(a)-6(e) are exemplary 2D HMI displacement maps.

FIG. 7 is a diagram illustrating normalized HMI displacement compared to depth in exemplary phantoms.

FIGS. 8(a)-8(e) are diagrams illustrating linear regression for determining attenuation in exemplary phantoms.

FIGS. 9(a)-9(c) are diagrams illustrating estimated attenuation compared to (a) independent measurement of exemplary phantoms, (b) Bland-Altman analyses of the measurements of (a), and (c) estimation errors of estimated attenuations with respect to independent measurement.

FIGS. 10(a1)-(b2) are diagrams illustrating HMI displacement depth and linear regression under different acoustic intensities.

FIGS. 11(a1)-(c3) are (a1)-(a3) HMI displacement maps and (b1)-(b3) HMI displacement curves obtained from three in vitro canine livers, and (c1)-(c3) linear regression for estimating attenuation of the livers.

FIG. 12 is a diagram illustrating estimated attenuations of liver tissues before and after ablation using different acoustic powers.

FIGS. 13(a)-13(c) illustrate exemplary HMI displacement maps, before HIFU exposure, after HIFU exposure, and after minus before (i.e., HMI displacement contrast map), respectively.

FIGS. 14(a)-14(f) illustrate exemplary HMI displacement maps and pathology images for purpose of comparison.

FIGS. 15(a)-15(d) are diagrams illustrating exemplary techniques for multi-parameter HMIFU according to the disclosed subject matter.

FIG. 16 is a diagram illustrating an exemplary HMIFU system for multi-parameter HMIFU according to the disclosed subject matter.

FIGS. 17(a)-17(d) are diagrams illustrating additional details of passive cavitation detection.

FIGS. 18(a)- 18(l) are diagrams illustrating additional details of multi-parameter HMIFU.

FIGS. 19(a)-19(f) are diagrams illustrating additional details of multi-parameter HMIFU.

FIGS. 20(a)-20(o) are diagrams illustrating additional details of multi-parameter HMIFU.

FIGS. 21(a)-21(o) are diagrams illustrating additional details of multi-parameter HMIFU.

FIG. 22 is a diagram illustrating additional details of multi-parameter HMIFU.

FIGS. 23(a)-23(f) are diagrams illustrating additional details of multi-parameter HMIFU.

FIGS. 24(a)-24(i) are diagrams illustrating additional details of multi-parameter HMIFU.

FIGS. 25(a)-25(i) are diagrams illustrating additional details of multi-parameter HMIFU.

FIG. 26 is a diagram illustrating an exemplary HMIFU system for HMIFU monitoring of slow denaturation and boiling HIFU treatment sequences according to the disclosed subject matter.

FIGS. 27(a)-27(i) are diagrams illustrating additional details of HMIFU monitoring of slow denaturation HIFU treatment.

FIGS. 28(a)-28(i) are diagrams illustrating additional details of HMIFU monitoring of boiling HIFU treatment.

FIGS. 29(a)-29(b) are diagrams illustrating additional details of HMIFU monitoring of boiling HIFU treatment and HMIFU monitoring of slow denaturation HIFU treatment for purpose of comparison.

FIGS. 30(a)-30(d) are diagrams illustrating additional details of HMIFU monitoring of boiling HIFU treatment and HMIFU monitoring of slow denaturation HIFU treatment for purpose of comparison.

FIGS. 31(a)-31(d) are diagrams illustrating additional details of HMIFU monitoring of boiling HIFU treatment and HMIFU monitoring of slow denaturation HIFU treatment for purpose of comparison.

Throughout the figures and specification the same reference numerals are used to indicate similar features and/or structures.

### DETAILED DESCRIPTION

The systems and methods described herein can be useful for estimating acoustic attenuation from time-varying radiation force information generated through the application of acoustic energy. Although the description provides as an example estimating acoustic attenuation of a biological system, such as biological tissue, the systems and methods herein can be useful for estimating acoustic attenuation of any suitable system that provides radiation force information through the application of acoustic energy.

The subject matter disclosed herein includes methods and systems for estimating acoustic attenuation in a tissue. Accordingly, they can utilize time-varying radiation force information generated through the application of acoustic energy to the tissue from at least first and second focal depths. An exemplary technique includes acquiring first signals representing oscillatory motion of the tissue in response to the radiation force proximate the first focal depth, and acquiring second signals representing oscillatory motion of the tissue in response to the radiation force proximate the second focal depth. The method can further include estimating the oscillatory motion of the tissue from each of the first and second signals, and estimating the acoustic attenuation in the tissue from the estimated oscillatory motion of the tissue from the first and second signals.

In accordance with the disclosed subject matter, estimating acoustic attenuation in a tissue can include estimating acoustic attenuation of biological tissues using HMI with a linear regression model. HMI can provide oscillatory information from displacement induced in a tissue, and resulting harmonics can be separated from quasi-static effects. Furthermore, HMI can provide a localized result at least in part because the displacement can be measured at the focus of the FUS transducer. In this manner, attenuation estimation using HMI can provide a quantitative technique for both elasticity imaging of soft tissue and assessment of tissue elasticity undergoing thermal ablation such as HIFU.

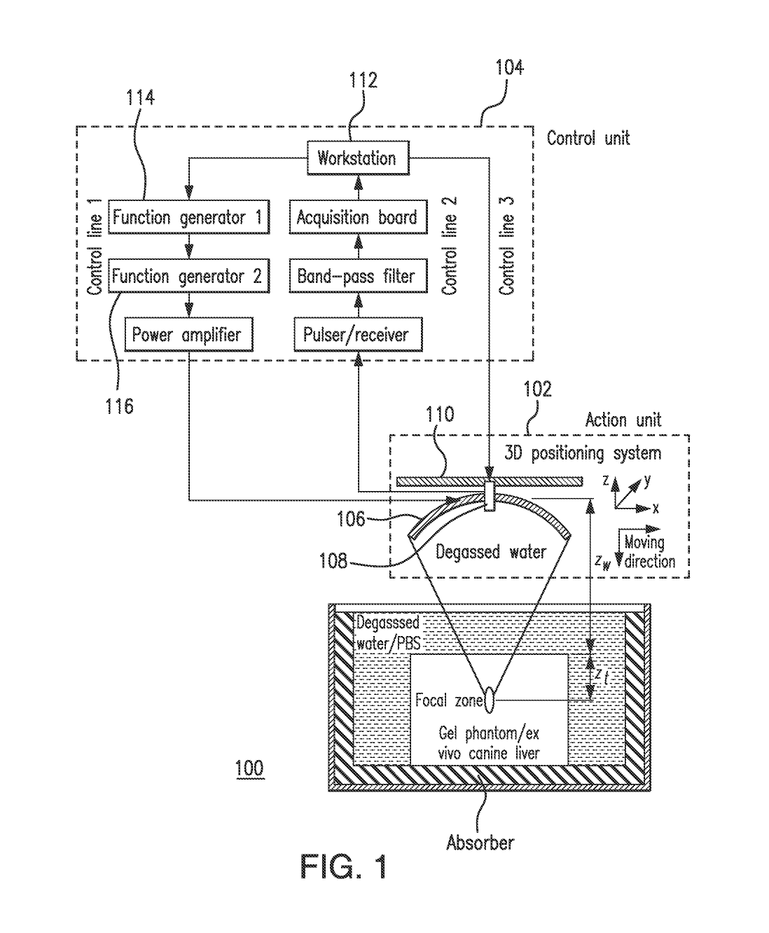

With reference to FIG. 1, an exemplary HMI system 100 according to the disclosed subject matter can generally include an action unit 102 and a control unit 104. The action unit 102 can include a focused ultrasound (FUS) transducer 106, for example and without limitation, embodied herein as a PZT transducer configured with a focal depth of 90 mm and center frequency (f_{center}=4.75 MHz), and an imaging transducer 108, for example and without limitation, a concentric and confocal single-element, pulse-echo transducer (embodied herein as a V30044 Immersion Transducer, focal depth: 6 cm, f_{center}=7.5 MHz , Olympus-NDT, Waltham, Mass., U.S.A.). FUS transducer 106 and imaging transducer 108 can be confocally aligned and attached to a 3D positioning system 110. Additionally or alternatively, as embodied herein, FUS transducer 106 and imaging transducer 108 can be coaxially-aligned. In addition, or as a further alternative, FUS transducer 106 and imaging transducer 108 can be joined together to facilitate joint movement of FUS transducer 106 and imaging transducer 108 in confocal and/or coaxial alignment. The control unit 104 can include a processor 112 operably coupled to the action unit 102. For example and without limitation, the processor 112 can be embodied as a PC workstation (CPU: 3.06 GHz; RAM: 80 GB), and can be operably coupled to the action unit by one or more control lines. For purpose of illustration and not limitation, three control lines are utilized from the processor 110, one for each of the FUS transducer 106 (also referred to as “Control line 1”), the imaging transducer 108 (also referred to as “Control line 2”), and the 3D positioning system 110 (also referred to as “Control line 3”).

For purpose of illustration, and as embodied herein, processor 112 can output an amplitude-modulated (AM) signal, for example and without limitation at a carrier frequency of 4.75 MHz, via Control Line 1 using a first signal generator 114 (also referred to as Function Generator 1, embodied herein as Model: 33220A, Agilent®, Calif., U.S.) and a modulation frequency, for example and without limitation at 25 Hz, via Control Line 1 using a second signal generator 116 (also referred to as Function Generator 2, embodied herein as Model: 33120A, HP®, NY, US). The activation duration of each signal can be 400 ms, and a duration between two adjacent bursts can be 1 s. The AM signal from signal generator 114 can be amplified through a RF power amplifier (for example, embodied here as Model: 3000L, ENI®, N.Y., U.S.A.), and thus can have an acoustic intensity of 0.1 W/cm² on the transducer surface. The acoustic intensity can be obtained by dividing the acoustic power (for example as measured using a radiation force balance) by the area of the active surface of the focused transducer (as embodied herein, the area of the transducer active surface=50.87 cm²). In this manner, the AM ultrasonic wave can induce a time-varying radiation force in the focal region of the FUS transducer, which can occur at twice the modulation frequency (i.e., 50 Hz). Oscillatory motion can thus occur at the focal zone, and can be detected by the imaging transducer 108 during force application.

Furthermore, and as embodied herein, the processor 110 can operate the imaging transducer 108 via Control line 2, for example in a pulse-echo manner using a pulser/receiver 118 (embodied herein as 5800PR, Olympus NDT®, N.Y., U.S.A.) for acquiring RF signals at a pulse repetition frequency (PRF), for example and without limitation at 4 kHz, and can occur in conjunction with the operation of Control line 1. The captured RF signals can be input into a band-pass filter for filtering out the carrier frequency, and can be digitized by a data acquisition board (Gage®, IL, U.S.A.), embodied herein with a sampling frequency of 100 MHz. 1D normalized cross correlation can be applied to the RF signals for estimating the oscillatory motion, embodied herein with a window size of 1 mm and 90% overlap.

In HMI according to the disclosed subject matter, the acoustic energy emitted by the FUS transducer 102 can converge at the transducer focus, and thus a radiation force can be locally generated, the magnitude of which can be represented as

$$F = \frac{2 α I}{c} , \qquad ( 1 )$$

where c (e.g., 1540 m/s), f, α=α(f) (dB/cm) and I (W/cm²) can represent the sound speed, frequency, frequency-dependent attenuation coefficient of the tissue and in situ temporal average intensity, respectively. The intensity (I) can be determined from the acoustic pressure (p) according to

$$I = \frac{{p}^{2}}{2 ρ c} , \qquad ( 2 )$$

where ρ can represent the density of the medium. The radiation force can be obtained from the acoustic pressure according to

$$F = \frac{α {p}^{2}}{ρ {c}^{2}} , \qquad ( 3 )$$

The activation surface of the FUS transducer can be represented as a concave spherical geometry modeled as a set of equivalent monopole sources uniformly distributed over the transducer aperture and excited in phase, and thus the pressure distribution of such a radiator can be approximated by Rayleigh function, for example in the form of an integral taken over the area of the transducer surface in a non-attenuating medium. The attenuation and dispersion effects associated with the transmission of the ultrasonic beam in an attenuating material can be represented as the complex wavenumber k_{c}, for example as

$${k}_{c} = \frac{2 π f}{c} + i α , \qquad ( 4 )$$

where i=\sqrt{−1}. As such, the pressure field at the focus in an attenuating homogeneous medium can be determined from eq. (4) as

$$\vert p ( R ) \vert = 2 {p}_{0} \frac{π {a}^{2}}{2 λ R} {e}^{- α R} , \qquad ( 5 )$$

where R, p₀, λ and α can represent the focal radius, acoustic pressure at the transducer surface, wavelength and transduce radius, respectively.

In the exemplary system and technique described herein, the wave propagation path can be considered to cover biphasic media: that is, water and tissue (i.e., an inhomogeneous medium). As such, the result of eq. (5) can be determined through the definition of a single medium using an effective attenuation coefficient without nonlinearity, which can be represented as

$${α}_{eff} = {α}_{t} ( \frac{{z}_{t}}{{z}_{w} + {z}_{t}} ) , \qquad ( 6 )$$

where z_{w} and zₜ can represent the propagation depths of the beam within the water and tissue, respectively. Furthermore, the attenuation of water can be relatively negligible.

Two different focal positions in the tissue can be represented with depth being respectively zₜ₁ (as illustrated in FIG. 2(a)) and zₜ₂ (as illustrated in FIG. 2(b)), and a reflection at the water-tissue interface can occur. As such, the ratio between the acoustic pressures at the foci of the two positions, as illustrated in FIGS. 2(a)-2(b), can be represented as

$$\frac{{p}_{1} ( z )}{{P}_{2} ( z )} = \frac{2 {t}_{1} {p}_{0} \frac{π {a}^{2}}{2 λ R} {e}^{- {α}_{eff 1} R}}{2 {t}_{2} {p}_{0} \frac{π {a}^{2}}{2 λ R} {e}^{- {α}_{eff 2} R}} = \frac{{t}_{1} {e}^{- {α}_{eff 1} R}}{{t}_{2} {e}^{- {α}_{eff 2} R}} = \frac{{t}_{1} {e}^{- {α}_{t} \frac{{z}_{t 1}}{{z}_{w 1} + {z}_{t 1}} R}}{{t}_{2} {e}^{- {α}_{t} \frac{{z}_{t 2}}{{z}_{w 2} + {z}_{t 2}} R}} , \qquad ( 7 )$$

where t₁, t₂ can represent the transmission coefficients between water and tissue in FIGS. 2(a) and 2(b), respectively, and α_{eff1}(f) and α_{eff2}(f) can represent the effective attenuations in FIGS. 2(a) and 2(b), respectively.

At the focus of the FUS transducer, for example where z_{w1}+zₜ₁=R (FIG. 2(a)) and z_{w2}+zₜ₂=R (FIG. 2(b)), eq. (7) can be represented as

$$\frac{{p}_{1} ( R )}{{P}_{2} ( R )} = \frac{{t}_{1} {e}^{- {α}_{t} {z}_{t 1}}}{{t}_{2} {e}^{- {α}_{t} {z}_{t 2}}} = \frac{{t}_{1}}{{t}_{2}} {e}^{- {α}_{t} ( {z}_{t 1} - {z}_{t 2} )} . \qquad ( 8 )$$

As discussed herein, α can represent the frequency-dependent attenuation coefficient of the tissue, i.e., α=α(f). The attenuation of the soft tissue can be linearly correlated with frequency as a first-order approximation, and thus αₜ(f)=αₜ f in eq. (8). The transmission coefficients t₁ and t₂ can be considered as identical, and as such, the two media (i.e., water and tissue) can remain the same in both cases, and the wave incident angle can change only insignificantly when zₜ₁ and z₂ are disposed a small distance apart, for example and without limitation, embodied herein as 5 mm. Furthermore, the radiation force (F) can change linearly based at least in part on the square of the acoustic pressure (eq. (3)). As such, the ratio between the radiation forces at depths zₜ₁ and zₜ₂ can be expressed by

$$\frac{{F}_{1} ( R )}{{F}_{2} ( R )} = {e}^{- 2 {α}_{t} f ( {z}_{t 1} - {z}_{t 2} )} . \qquad ( 9 )$$

The attenuation coefficient can thus be obtained, for example by

$${α}_{t} = \frac{ln ( \frac{{F}_{1} ( R )}{{F}_{2} ( R )} )}{2 f ( {z}_{t 2} - {z}_{t 1} )} . \qquad ( 10 )$$

As such, the acoustic attenuation can be obtained from the ratio between F₁(R) and F₂(R), frequency (f), and distance between zₜ₁ and zₜ₂. The radiation force at the focus of the FUS transducer (i.e., F₁(R) and F₂(R)) can be obtained from testing samples by varying the output intensity of the FUS transducer (embodied herein as 0.03-0.22 W/cm²), and represented as I=k′D, where D can represent HMI displacement, I can represent output intensity of the FUS transducer and k′ can represent a linear coefficient, as discussed further herein. Such an examination can be performed at multiple, pre-selected focal positions covering the whole raster-scan plane in the sample, for example and embodied herein using 5 positions at each focal depth. Furthermore, the intensity and the induced radiation force can be linearly proportional at certain frequencies, for example the HMI carrier frequency utilized herein, and thus can be represented as F=k″I, where F can represent output intensity of the FUS transducer, and k″ linear coefficient, which can be determined from equation (1). The ratio between the radiation forces can thus be represented as equal to that of displacements, that is

$$\frac{{F}_{1} ( R )}{{F}_{2} ( R )} = \frac{{D}_{1}}{{D}_{2}} , \qquad ( 11 )$$

where D₁ and D₂ can represent displacements induced by F₁ and F₂ at depths zₜ₁ and zₜ₂, respectively. As such, HMI-related acoustic attenuation can be represented as

$${α}_{t} = \frac{ln ( \frac{{D}_{1}}{{D}_{2}} )}{2 f ( {z}_{t 2} - {z}_{t 1} )} . \qquad ( 12 )$$

The technique for obtaining the representation of eq. (12) can be applied to the HMI displacements estimated at different depths for attenuation estimation, and thus the displacement at every depth (D_{z}) can be compared with that at the initial depth (D₀), which can be represented as

$${α}_{t} = \frac{ln ( \frac{{D}_{0}}{{D}_{z}} )}{2 f ( z - {z}_{0} )} , \qquad ( 13 )$$

where z₀, z, D₀ and D_{z} can represent the initial depth, the arbitrary depth, the HMI displacements at the initial depth and arbitrary depth, respectively. In this manner, the attenuation can be estimated using a linear regression model, for example and as embodied herein, by linearly correlating

$$\frac{ln ( \frac{{D}_{0}}{{D}_{t}} )}{2 f}$$

and (z−z₀). Such a technique can thus utilize differences in HMI displacement at different focal depths, where attenuation effect can be a factor in the decrease in acoustic energy when the focus deepens.

The techniques described herein can be applied, for purpose of illustration and confirmation of the disclosed subject matter, and not limitation, to estimate attenuation in five phantoms with known attenuations (Computerized Imaging Reference Systems (CIRS), Inc., VA, U.S.A.) (as shown in Table 1). The phantoms can include three normal canine livers in vitro and five canine livers in vitro after HIFU ablation. The phantoms, for illustration and not limitation, and as embodied herein, can have dimensions of 50 mm in diameter and 50 mm in height, and can have homogeneous material properties. The attenuation of each phantom can be measured using log spectral difference measurement, with the parameters listed in Table 1. Each phantom can be immersed in degassed water in a water tank during the measurement with the phantom sealed using a thin membrane to avoid water ingress. Rubber absorbers can be placed between the phantom and edges of the water tank to avoid reflections of the ultrasound waves, as illustrated for example in FIG. 1. The phantoms can be tested using HMI, as described herein, with the confocally-aligned FUS transducer 106 and imaging transducer 108 operated in a raster-scan format, for example at a scanning step of 0.5 mm with the total scanned area of 5×5 mm² in the y-z plane. The scanned region can be chosen to be at least 3 mm deep from the upper surface of the phantom to avoid any boundary effects.

TABLE 1
Acoustical and mechanical properties of five CIRS phantoms
for attenuation measurement (provided by the producer)

| Sample No. | 1 | 2 | 3 | 4 | 5 |
|---|---|---|---|---|---|
| SOS (m/s) | 1542 | 1543 | 1540 | 1538 | 1540 |
| Attenuation | 0.28 | 0.57 | 0.90 | 1.17 | 1.45 |
| (dB/cm/MHz) | | | | | |
| Backscatter intensity | 0.13 | 0.13 | 0.10 | 0.08 | 0.05 |
| (arbitrary units) | | | | | |
| Contrast (dB) | 0 | 0 | 1.14 | 1.9 | 4.15 |
| Attenuation adjusted | 0 | 1.1 | 1.22 | 1.49 | 0.30 |
| contrast (dB) | | | | | |
| Elasticity (kPa) | 6 | 5 | 5 | 4 | 5 |

Furthermore, for purpose of illustration and confirmation of the disclosed subject matter, to determine the effect of the output acoustic intensity of the FUS transducer on the results, two additional examples can be conducted (herein on Phantom 2), embodied herein with (i) the acoustic intensity at 0.1 W/cm² (as performed above) and the raster scanning depth of 10 mm at a scanning step of 1 mm; and (ii) with the same configuration described herein but with the acoustic intensity increased to 0.2 W/cm². As embodied herein, Phantom 2 can have an attenuation of 0.57 dB/cm/MHz, which can represent an attenuation of biological tissues.

Additionally, for purpose of illustration and confirmation of the disclosed subject matter, the techniques described herein were applied to estimate the attenuations of three in vitro canine livers obtained from three mongrel male dogs. Each specimen was immersed in phosphate buffered saline (PBS) solution and placed in a vacuum chamber for one and a half hours for degassing. The liver tissues were moved from the vacuum chamber to the water bath filled with degassed PBS solution, and the samples remained submerged in degassed saline to avoid air exposure. The attenuation measurement in the liver remained the same to that of phantoms.

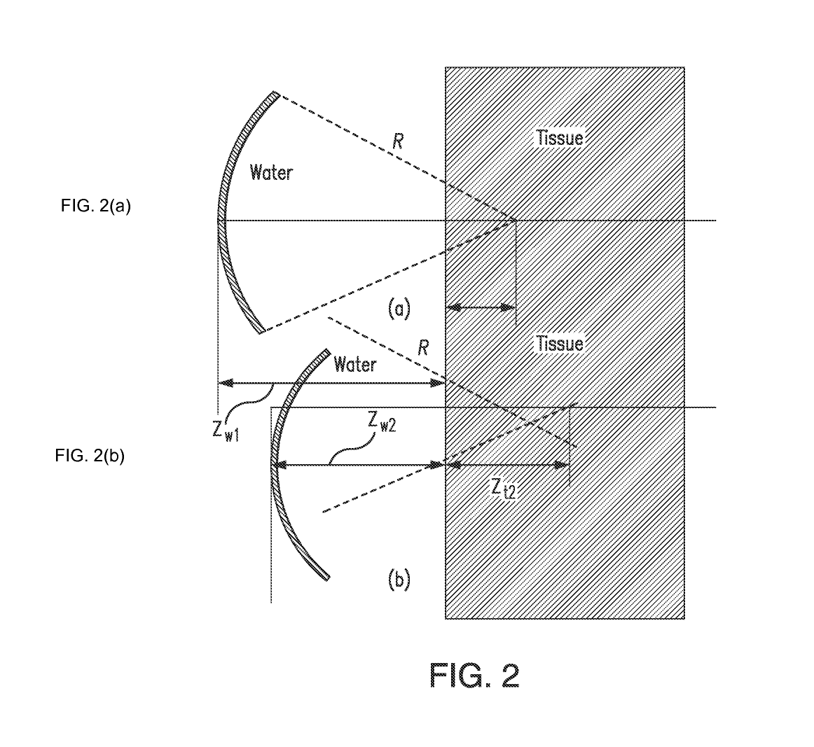

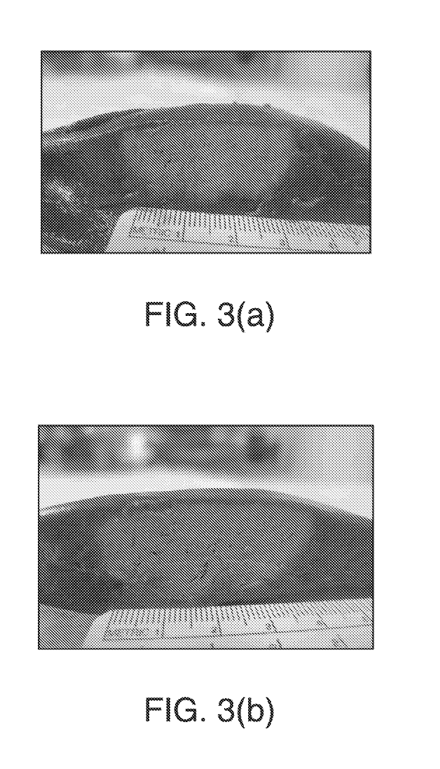

In addition, for purpose of illustration and confirmation of the disclosed subject matter, five in vitro canine livers were ablated using the FUS transducer 102 excited (i) at 600 mV (using Function generator 1, providing an acoustic intensity of about 0.1 W/cm² at the surface of the FUS transducer) with an activation duration of 120 s, or (ii) at 900 mV (using Function generator 1, providing an acoustic power of around 0.21 W/cm² at the surface of the FUS transducer) for 30 s. In these examples, the FUS transducer was operated in a raster-scan manner, i.e., with 11 consecutive positions moved sequentially by 3 mm in the lateral direction and 2 positions offset by 3 mm in the axial direction, providing a lesion with the dimension of roughly 2×3 cm², as illustrated in FIG. 3(a) for acoustic intensity of 0.1 W/cm² and FIG. 3(b) for acoustic intensity of 0.21 W/cm². The attenuation of each liver was measured before ablation, to provide a reference, and two different acoustic powers were chosen to determine the effect of different acoustic intensities during HIFU ablation on the attenuation of the induced lesions.

### EXAMPLE 1

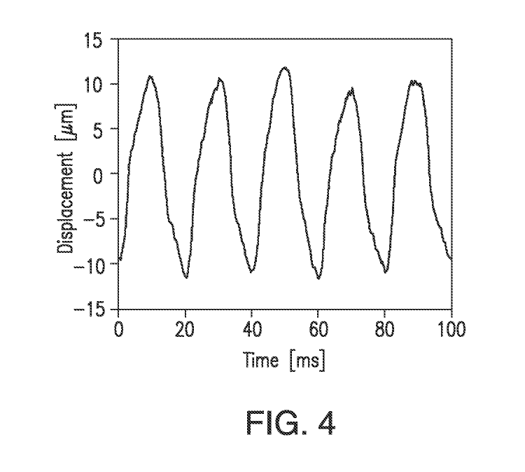

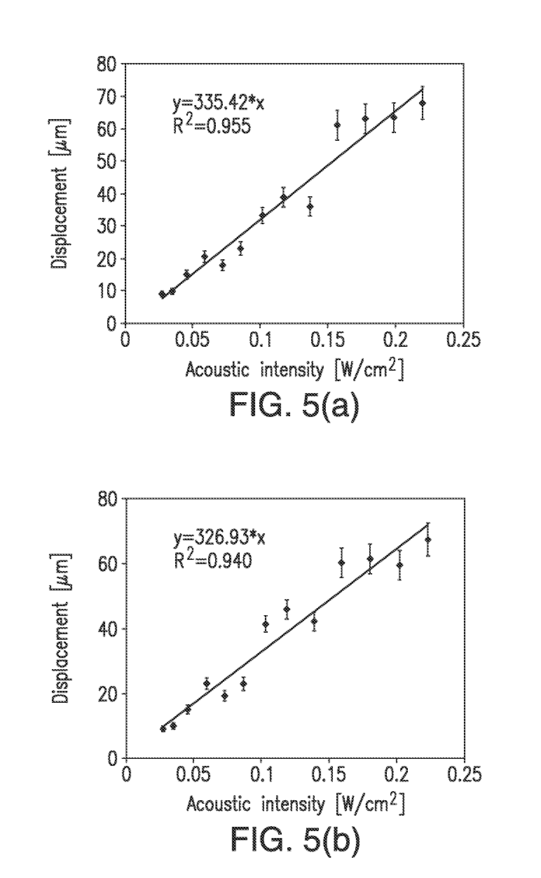

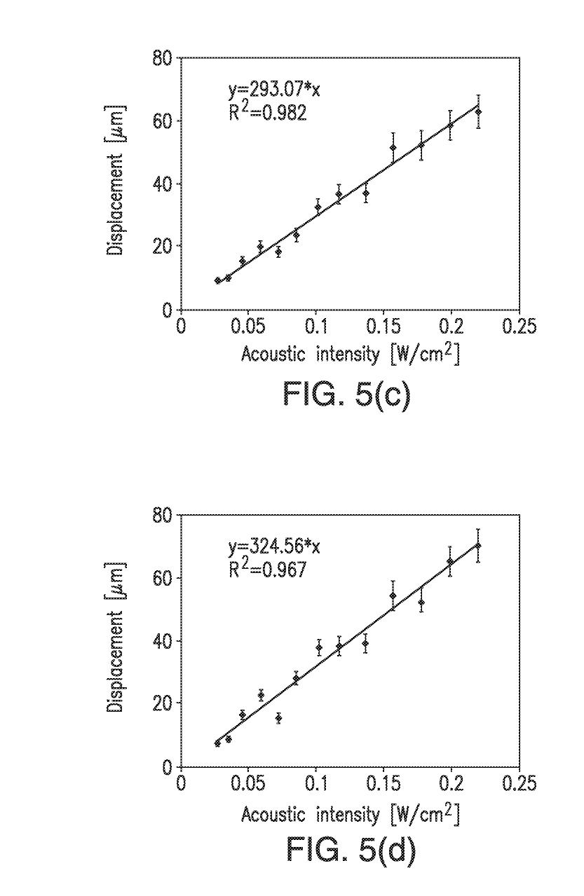

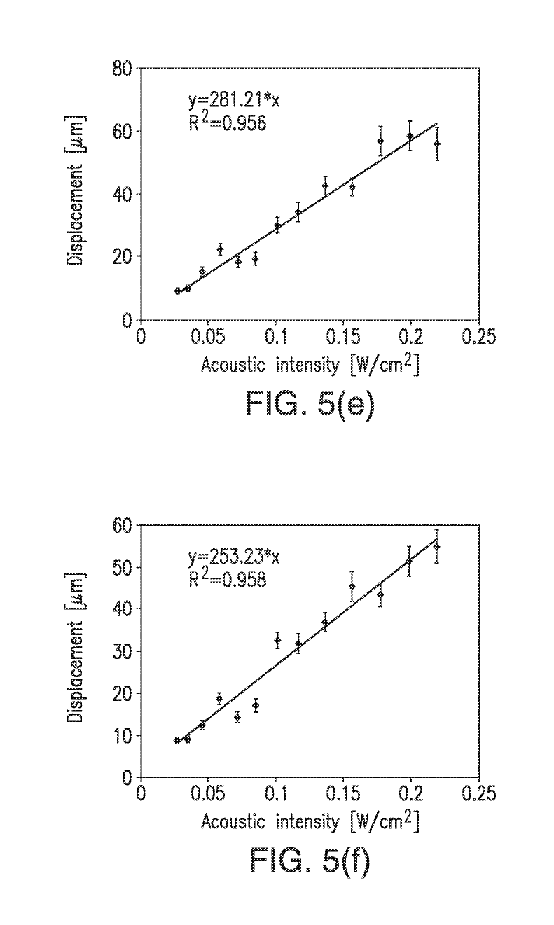

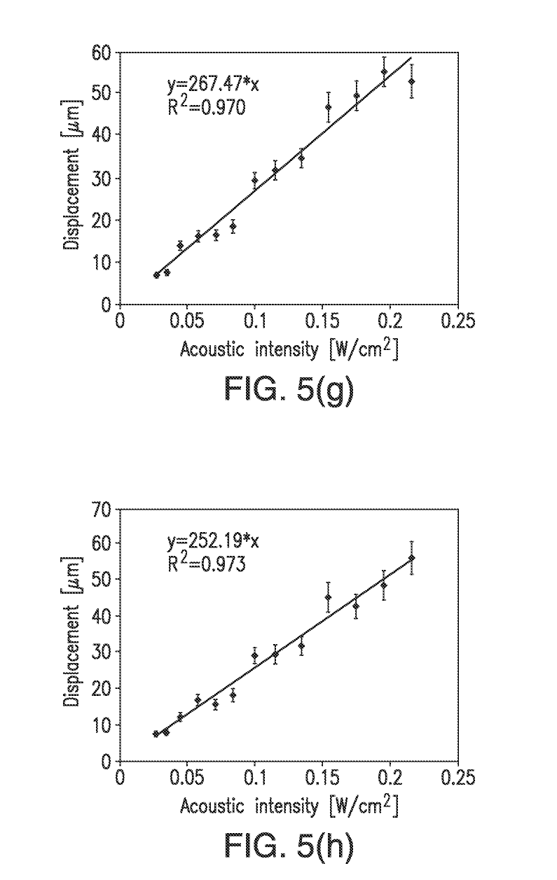

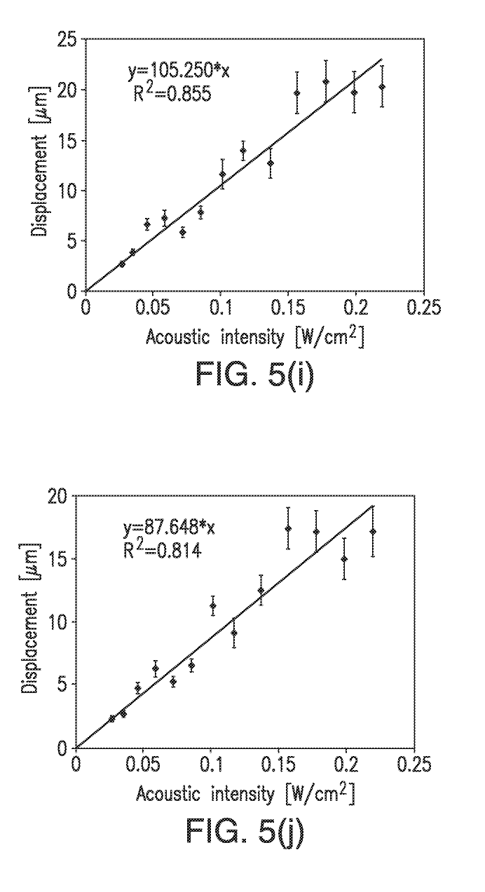

As a representative example, FIG. 4 illustrates the displacement curve at the focus of the FUS transducer over 100 ms, embodied herein with an output intensity at the transducer surface of 0.1 W/cm², captured from Phantom 1. As discussed herein, the linear elasticity of each sample was evaluated by varying the output acoustic intensity of the FUS transducer. The relationship between the acoustic intensity and induced displacement in the five phantoms, three in vitro normal livers and five livers with HIFU lesions is illustrated in FIG. 5(a)-5(j), (FIGS, 5(a)-5(e) for phantoms 1-5, FIGS. 5(f)-5(h) for three liver samples and FIGS. 5(i)-5(j) for HIFU lesions produced using two different acoustic intensities, respectively), with the correlation coefficient varying within 0.814-0.982.

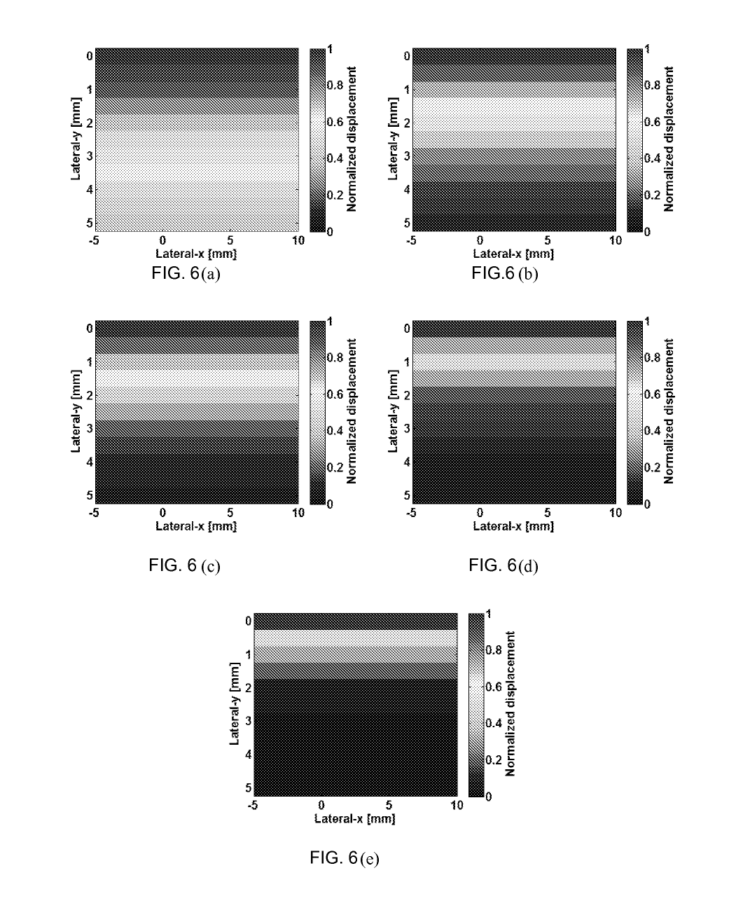

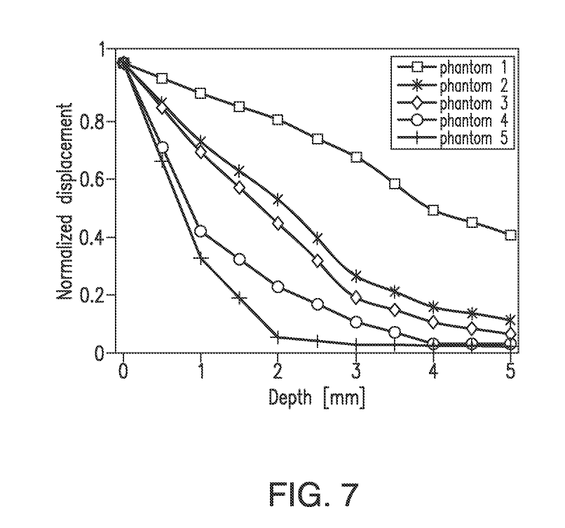

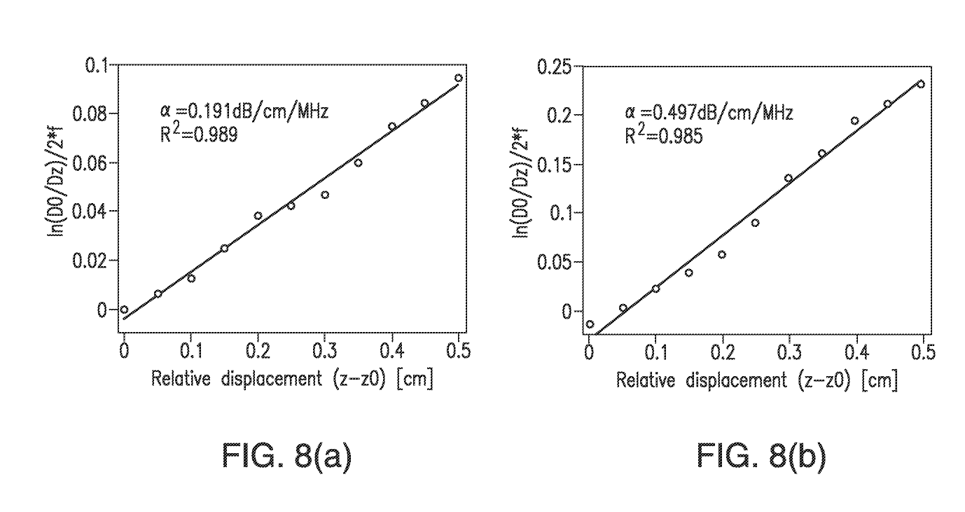

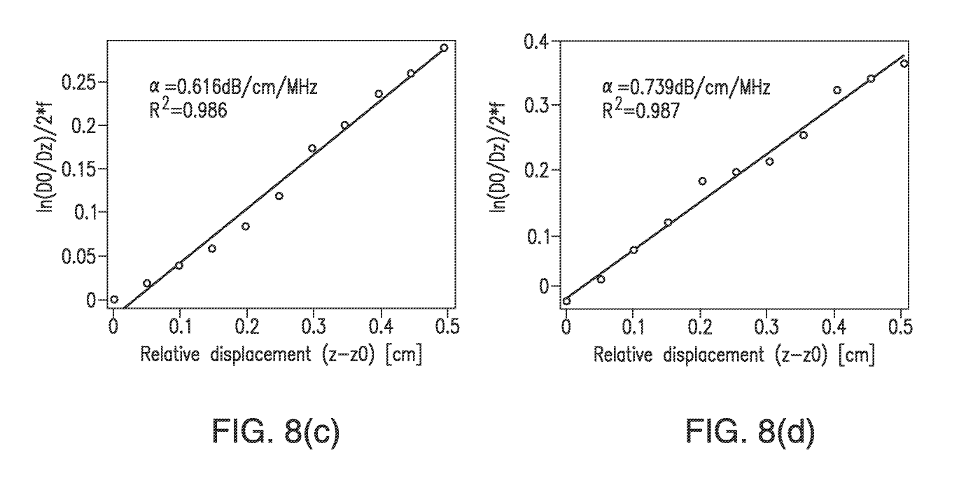

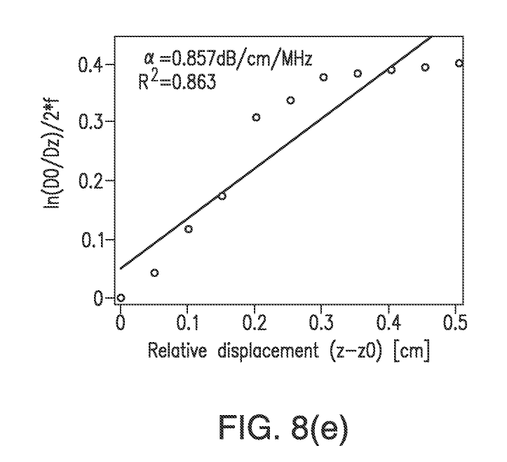

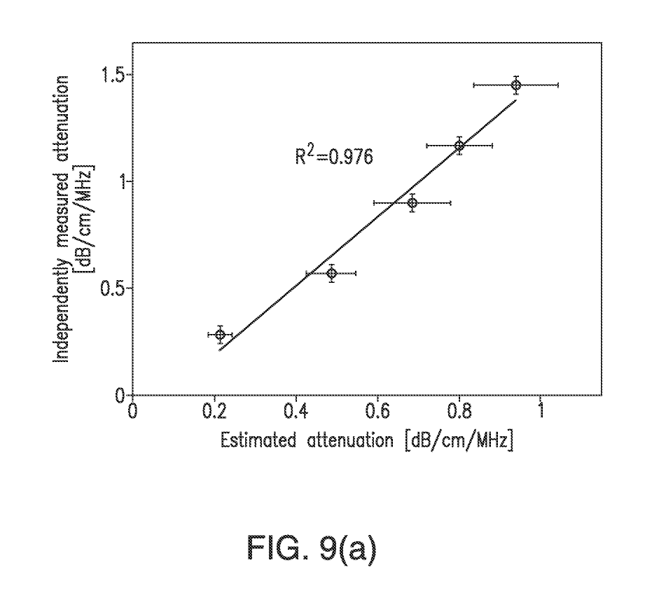

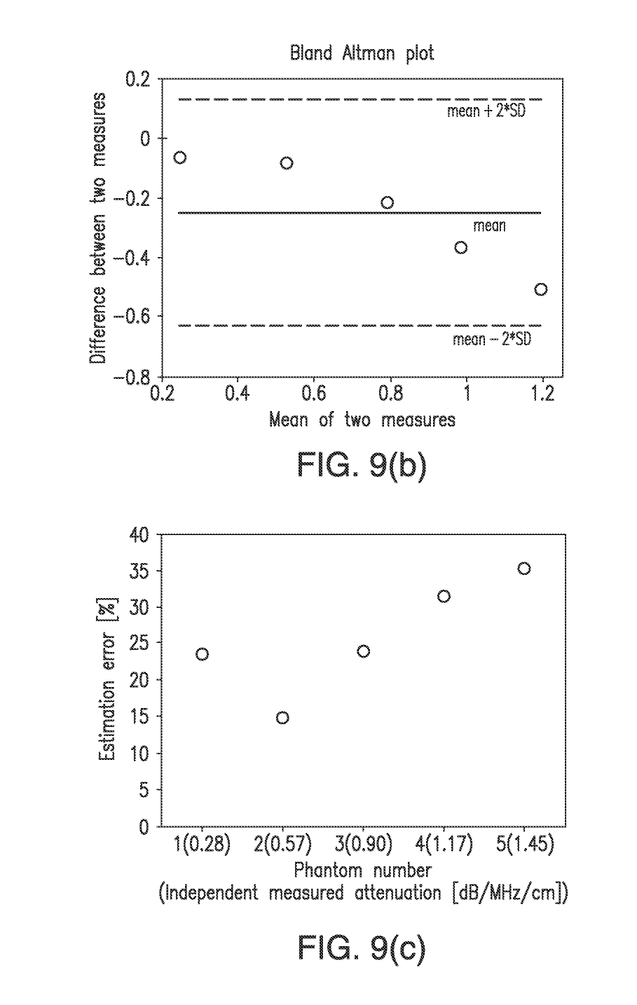

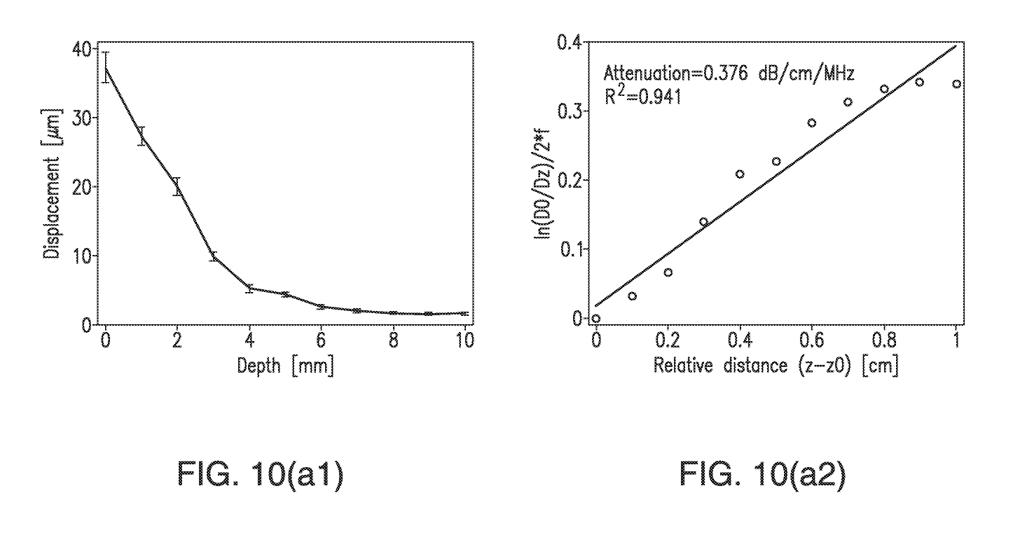

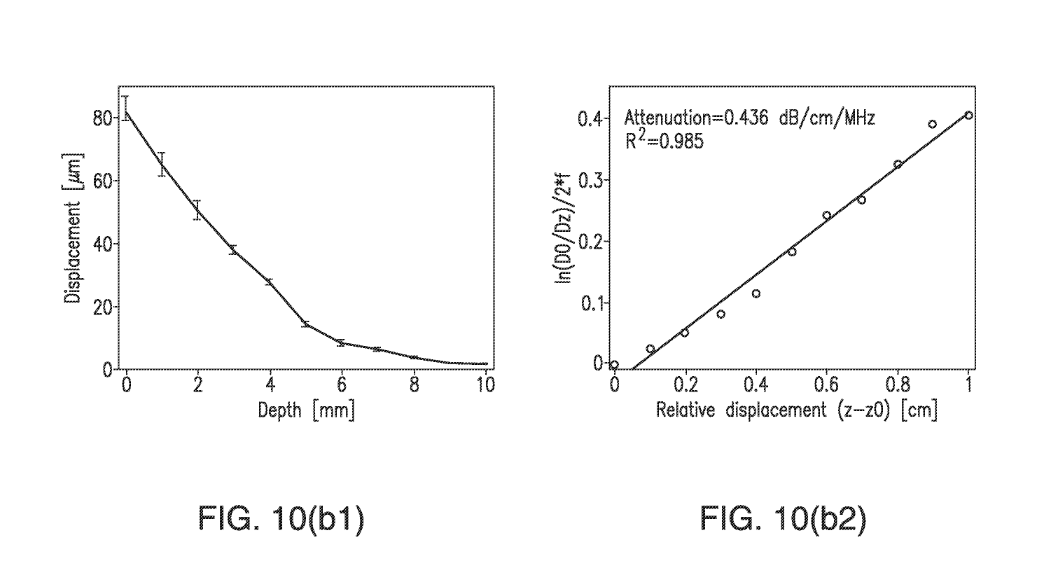

The HMI displacements were estimated in all raster-scan locations, forming a 2D HMI displacement map, as shown for example in FIG. 6(a)-6(e) (for phantoms 1-5). The average HMI displacement, which can correspond to the average peak-to-peak HMI displacements over the duration of the HMI application, as shown for example in FIG. 4, was obtained at different depths and each compared in FIG. 7. FIGS. 8(a)-8(e) illustrate the estimated attenuations of phantoms 1-5 using the linear regression model (i.e., equation (13)), respectively, which are also listed in Table 1. The estimated attenuations were compared with those independently measured (from Table 1), with the correlation coefficient equal to 0.976 (as illustrated in FIG. 9(a)). FIG. 9(b) illustrates a Bland-Altman analysis of the data, and FIG. 9(c) illustrates estimation errors that varied within 15%-35%. FIG. 10(a1)-10(b2) shows the estimated displacements and attenuations using different acoustic intensities (i.e., 0.1 and 0.2 W/cm²) to illustrate effects of the output acoustic intensity of the FUS transducer on the techniques of the disclosed subject matter.

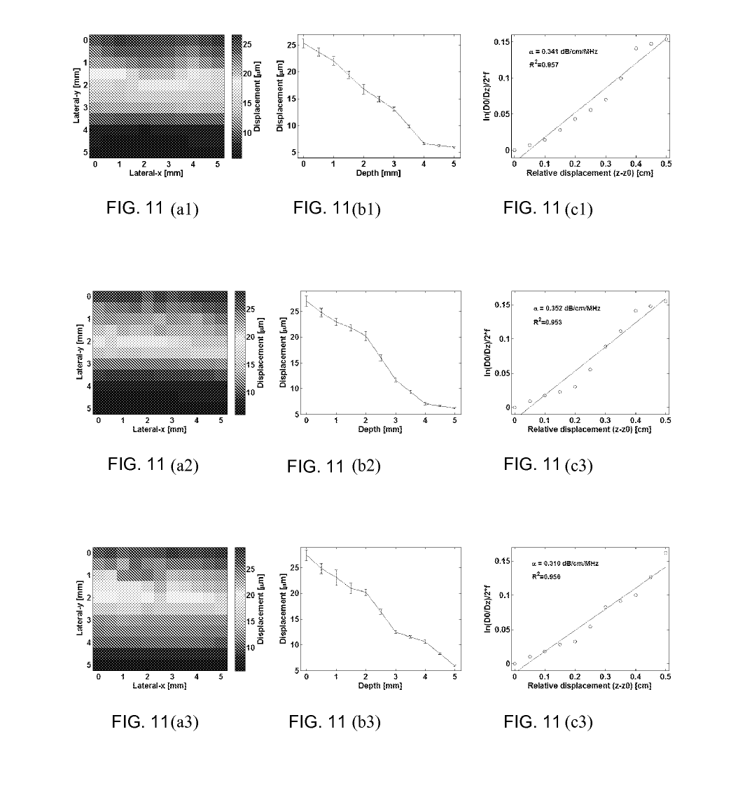

The attenuation in three in vitro canine livers, as shown for example in Table 2, varied in a range from 0.293 to 0.353 dB/cm/MHz. For purpose of illustration, FIG. 11(a1)-11(c3) presents the displacement maps (FIGS. 11(a1)-11(a3)) and plots (FIGS. 11(b1)-11(b3)) obtained from three in vitro canine livers, from which the estimated attenuations are illustrated in FIGS. 11(c1)-11(c3), respectively.

TABLE 2
Estimated attenuations of five CIRS phantoms (unit: dB/cm/MHz)

| Phantom | | | | | | | |
|---|---|---|---|---|---|---|---|
| No. | Test-1 | Test-2 | Test-3 | Test-4 | Test-5 | Mean | SD |
| 1 | 0.176 | 0.193 | 0.224 | 0.255 | 0.221 | 0.214 | 0.031 |
| 2 | 0.573 | 0.503 | 0.495 | 0.424 | 0.435 | 0.486 | 0.06 |
| 3 | 0.78 | 0.787 | 0.618 | 0.58 | 0.659 | 0.685 | 0.944 |
| 4 | 0.857 | 0.776 | 0.834 | 0.869 | 0.67 | 0.801 | 0.082 |
| 5 | 1.092 | 0.974 | 0.95 | 0.852 | 0.836 | 0.941 | 0.104 |

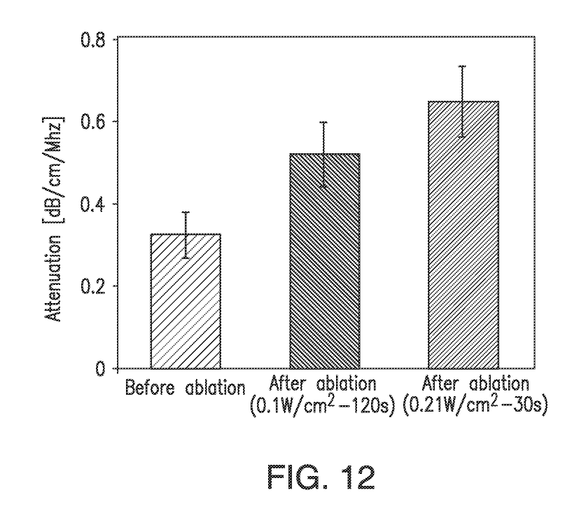

The estimated attenuations in in vitro canine livers before and after ablation, i.e., HIFU lesions, are illustrated, for purpose of comparison, in FIG. 12 and Table 3. Paired-sample t-test evaluation showed differences in estimated attenuation between the normal tissue and HIFU lesions using different acoustic intensities, that is, p-values of 0.0018 for the tissue before and after ablation at acoustic intensity of 0.1 W/cm², 1.06×10⁻⁴ for the tissue before and after ablation at acoustic intensity of 0.21 W/cm² and 0.0383 for HIFU lesions using different acoustic intensities. As such, the HIFU lesions were measured to have higher attenuation than the normal tissues, and of the HIFU lesions, those produced using higher acoustic intensity were estimated to have higher attenuation.

TABLE 3
Estimated attenuations of three in vitro canine livers (unit: dB/cm/MHz)

| Phantom | | | | | | | |
|---|---|---|---|---|---|---|---|
| No. | Test-1 | Test-2 | Test-3 | Test-4 | Test-5 | Mean | SD |
| 1 | 0.282 | 0.292 | 0.323 | 0.367 | 0.352 | 0.323 | 0.037 |
| 2 | 0.412 | 0.364 | 0.341 | 0.31 | 0.338 | 0.353 | 0.038 |
| 3 | 0.341 | 0.318 | 0.273 | 0.263 | 0.276 | 0.294 | 0.034 |

Attenuation estimation using HMI, according to the disclosed subject matter, can be performed to simultaneously generate the radiation force and monitor the induced local displacement at the focus of the FUS transducer. The HMI displacements estimated at different depths within the raster-scan plane can be analyzed using a linear regression model for estimating the attenuation. In this manner, the local displacement (i.e., HMI displacement) can vary with depth, and thus localized tissue attenuation within a region can be estimated. As such, the techniques according to the disclosed subject matter can be applied to evaluate tissues with regional inhomogeneity, for example and without limitation, tumors and HIFU-induced thermal lesions. Furthermore, as discussed herein, the techniques can be performed independent of the speed of sound, as shown for example in eqs. (3) and (9), and as such, tissues can be evaluated under thermal treatment.

In the examples discussed herein, the HMI displacements were estimated at all the raster-scan locations to form the 2D HMI displacement maps, as shown for example in FIG. 6(a)-6(e). In the phantoms, in vitro livers and HIFU lesions of the examples, a linear relationship between the acoustic intensity and HMI displacement was determined, as illustrated for example in FIG. 5(a)-5(j). Such a relationship was determined for all sample points selected throughout the raster-scan plane. As such, the samples were considered as linearly elastic at the force amplitude and frequency used. In addition, soft tissues can generally be considered as linearly elastic at low excitation frequencies (e.g., less than 100 kHz), and the tissue harmonic motion induced by the radiation force, as embodied herein, was determined to be 50 Hz, which further supports the linear relationship of the displacements.

Referring now to FIG. 6(a)-6(e), the HMI displacement decreased with depth, due at least in part to the effect of attenuation. The decreasing rates in HMI displacement were different for phantoms of different attenuations, that is, for phantoms of higher attenuations, the HMI displacements decrease at a higher rate, as illustrated for example in FIG. 7. As such, the attenuations of those phantoms were differentiated using the linear regression model of eq. (13), as shown for example in FIG. 8(a)-8(e) having high correlation coefficients. With reference to FIG. 8(a)-8(e), the correlation coefficient for Phantom 5 (0.863) with the higher attenuation is lower than those for Phantoms 1-4 (about 0.98). The correlation coefficients can be due, at least in part, to an HMI displacement decreasing rapidly with depth for the phantoms with relatively high attenuation, as illustrated in FIG. 7. As a result, the signal-to-noise ratio (SNR) of the technique decreased.

In human soft tissues, attenuation can relate to various pathological conditions, as discussed above. The attenuations estimated using the proposed technique were found to linearly correlate with those independently measured, as shown for example in FIG. 9(a), with a linear correlation coefficient of 0.976, and analyzed by Bland-Altman analysis, as shown for example in FIG. 9(b). The estimation errors are illustrated in FIG. 9(c), and vary within the range of about 15%-35%. Furthermore, the phantom with the highest attenuation (e.g., Phantom 5) exhibited the largest estimation error. The estimation error can be due, at least in part, to the displacement in the high attenuated phantom decreasing quickly with depth. Furthermore, attenuated displacements beyond certain depths can introduce noise at least in part due to resolution limits of the equipment, and as such, the noise can deteriorate the estimated value and introduce estimation errors and reduce SNR. The estimation errors are relatively smaller for phantoms of low attenuations (e.g., Phantoms 1 and 2), which can represent superficial tissue regions or other tissues with lower attenuations, e.g., in vivo livers. Additionally, SNR can be increased by raising the acoustic intensity, which can result in higher HMI displacement in higher attenuating materials. With reference to FIG. 10(a1)-10(b2), the estimation using the higher acoustic intensity has a higher regression coefficient (0.985), and the attenuation value (0.436 dB/cm/MHz) is closer to that independently measured (0.57 dB/cm/MHz in Table 1) than the estimate using the lower acoustic intensity. As such, the estimation accuracy and sensitivities in high attenuating materials can be improved by increasing the acoustic intensity.

Furthermore, the higher intensity (i.e., 0.2 W/cm²) remained within the range in which the phantoms can be tested and deemed to be linearly elastic, as shown for example in FIGS. 5(a)-5(j), and thus eq. (13) can be applied to the attenuation estimation. With reference to FIG. 10(a2), the attenuation obtained using the higher intensity (i.e., 0.436 dB/cm/MHz) was compared with that using the low intensity (i.e., 0.376 dB/cm/MHz) to illustrate the effect of the estimated attenuation on the acoustic intensity. The accuracy of the estimated attenuation at higher acoustic intensities can be affected at least in part by thermal effects under higher acoustic intensities. This can be due at least in part because the tissue stiffness can be changed, and thus the HMI displacements can be altered. Furthermore, the tissue attenuation can be temperature-dependent. As discussed herein, the HMI sequence can be configured to produce displacement measurement with suitable SNR while reducing or minimizing any thermal/nonlinear effects.

Referring now to FIGS. 11(a1)-11(a3) and 11(b1)-11(b3), HMI displacement maps and curves, respectively, from three in vitro canine livers are illustrated. A decreasing relationship of the HMI displacement with depth can be observed, similar to that of the phantoms in FIGS. 6(a)-6(e) and 7. The corresponding linear regression results are illustrated in FIGS. 11(c1)-11(c3), with the linear regression coefficient being around 0.95, which can indicate that the liver is relatively homogeneous. The estimated acoustic attenuation of the canine livers was determined to be 0.32±0.03 dB/cm/MHz (as shown in Table 2) using the techniques of the disclosed subject matter, which corresponds to suitable ranges for in vitro normal livers (e.g., 0.28-0.399 dB/cm/MHz).

The phantoms used in this study were of homogeneous material property, which can correspond to the high linear regression coefficients, as shown in FIG. 7. For the in vitro livers tested in this study, the homogeneity likewise corresponds to linear regression coefficients of around 0.95, as shown for example in FIGS. 11(c1)-11(c3). The HMI displacement can be considered a local measurement, and thus inhomogeneity can introduce undesired bias to the estimation results. Increasing the raster-scan density, e.g., reducing the scanning differential, can reduce the effect of such bias, and can be used to provide an attenuation map of inhomogeneous tissues.

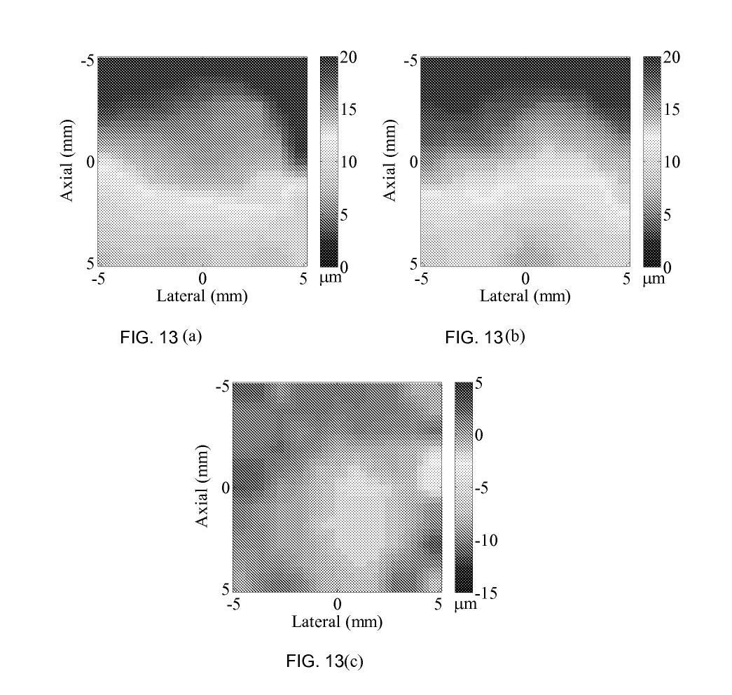

Referring now to FIG. 12, the HIFU lesions had higher attenuation than the normal tissues. Furthermore, the HIFU lesion produced under the acoustic intensity of 0.21 W/cm² was shown to have higher attenuation compared to those under the intensity of 0.1 W/cm², due at least in part because the higher intensity can change the property of the tissue more severely and increase attenuation. As such, the techniques according to the disclosed subject matter can be performed on inhomogeneous tissues, e.g., HIFU lesions, as well as homogeneous tissues. FIGS. 13(a)-13(c) illustrate the exemplary qualitative attenuation maps before (FIG. 13(a)) and after (FIG. 13(b)) HIFU ablation in a canine liver, as well as the difference between the before and after maps (FIG. 13(c)), which can highlight the location of lesions.

With reference to FIGS. 13(a)-13(c), qualitative attenuation maps, which can be referred to as 2D-HMI displacement maps, can be formed by, at each acquisition point during a raster scan, acquiring a M-mode including 600 RF lines using a single-element, pulse-echo transducer at a frame rate of 5.4 kHz. Displacements can be estimated using a 1-D normalized cross-correlation (e.g., window size of 1 mm and 85% overlap) technique on the received RF signals. A third-order median filter can be applied with bilinear interpolation on the displacement map to enhance the image resolution. For example and as embodied herein, stiffness increased with HIFU exposure, and as such a decrease of displacement occurred within the thermal lesion.

Additionally or alternatively, specimens can be sectioned along the raster scan plane following ablation and gross pathology images can be acquired and thresholded in contrast and brightness using an image manipulation tool (e.g., Microsoft Office® Picture Manager) to enhance the visualization of the formed lesions.

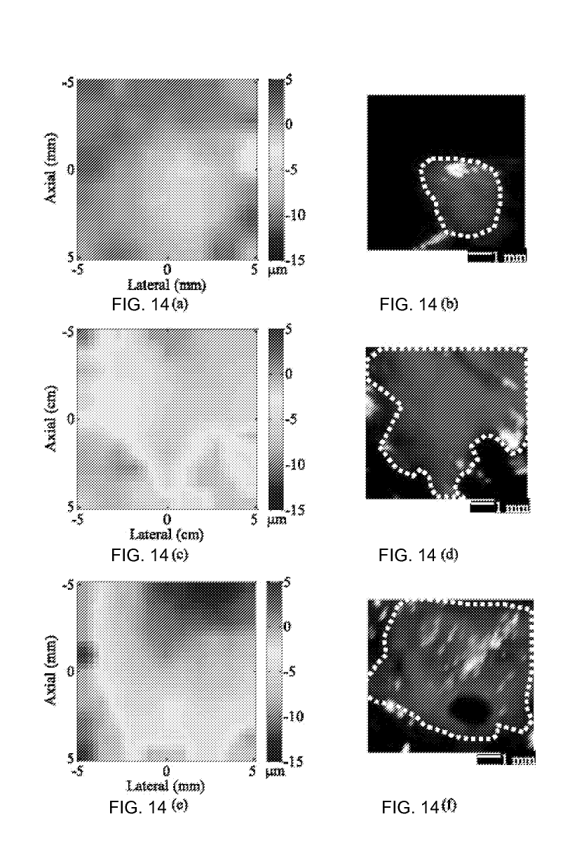

With reference to FIGS. 14(a)-14(f), HMI displacement contrast maps can be compared to gross pathology for purpose of comparison. For example, FIGS. 14(a), 14(c) and 14(e) illustrate a displacement contrast map, as described herein, of a lesion formed under 10 seconds, 20 seconds and 30 seconds, respectively, of HIFU exposure, side-by-side for purpose of comparison with FIGS. 14(b), 14(d) and 14(f), respectively, showing a gross pathology image of the same lesion. Contour lines have been added to the gross pathology images of the lesions to facilitate depiction of the induced lesion. As shown, the HMI lesion-to-background displacement contrast can linearly increase with the treatment time, which can indicate a positive correlation between stiffness and HIFU exposure duration.

As embodied herein, HMIFU can detect HIFU lesions based from a change in stiffness and can accurately depict an increase in HIFU lesion size at distinct exposure durations. In this manner, HMI can be used, for example and without limitation, in inducing a lesion and detecting the onset of coagulation at different treatment durations based on a change in elasticity, additionally or alternatively, quantifying the increase in lesion size with respect to treatment duration.

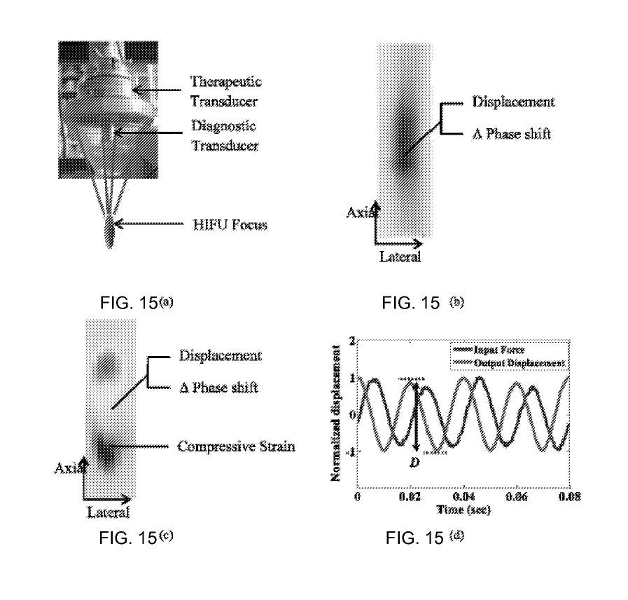

According to another aspect of the disclosed subject matter, multi-parametric HIFU monitoring can be performed. For example and without limitation, and as embodied herein, amplitude-modulated HIFU beams induce a sinusoidal displacement profile at the geometric focus, as described herein. The motion can originate from the acoustic radiation force generated due to the energy absorption from the HIFU beam, as described herein with respect to eq. (1). The relationship between induced displacement and excitation force can be described as wave propagation within a linear elastic medium, as follows:

$$ρ \frac{{\partial }^{2} u}{{\partial }^{2} t} = ( K + \frac{μ}{3} ) ∇ ( ∇ \cdot u ) + μ ∇ u + ρ F , \qquad ( 14 )$$

where ρ can represent the density of the medium, K can represent the bulk modulus, μ can represent the sheer modulus, F can represent the volumetric force, and u can present the induced displacement. For purpose of illustration and not limitation, as embodied herein, displacement along the propagation direction (z) is utilized, such that F=F(z), and μ(z). As embodied herein, an oscillatory response can be induced at the HIFU focal zone, which can be due to AM-HIFU excitation, as shown for example in FIG. 15(d). Although the displacement can also arise from thermal expansion and variation in speed of sound, such displacement can be considered as much smaller than oscillatory mechanical motion from acoustic radiation force from AM-HIFU beam using HMI. Such HMI displacement can be monitored throughout the HIFU treatment duration. The relative change of peak-to-peak HMI displacement amplitude with respect to displacement at a baseline, for example at the beginning of HIFU treatment, can be correlated with the relative change in local tissue stiffness as the thermal lesion develops. As embodied herein, the displacement parameter can be utilized. Displacement can be considered a qualitative parameter, which can account for changes in acoustic and mechanical properties at the focus during HIFU treatment. For example and without limitation, displacement can be less clear at revealing relative changes in tissue stiffness occurring along with increase of acoustic absorption, e.g., increased displacement amplitude. As such, complementary approaches can be utilized to increase reliability.

With reference to FIGS. 15(a)-15(d), within the acoustic focal zone where displacement can be monitored, another parameter, phase shift, can also be monitored. The phase shift can refer to the phase angle difference between the applied force and induced displacement profile. In the frequency domain, the complex modulus can be derived from calculating the ratio between the oscillatory excitation force and the induced displacement with a delay φ,

$$\frac{σ ( ω )}{ɛ ( ω )} = \frac{{σ}_{0} {e}^{i ω t}}{{ɛ}_{0} {e}^{i ( ω t - ϕ )}} = {G}^{′} + i {G}^{″} , \qquad ( 15 )$$

where σ₀ can represent the pressure amplitude, ω can represent the modulation frequency, ε₀ can represent the strain amplitude, i²=−1, G′ can represent the shear storage modulus, G 41 can represent the shear loss modulus, φ can represent the phase angle between force and displacement profile, and t can represent the time. The phase angle between these two functions can be represented as the ratio between G′ (elasticity) and G″ (viscosity).

$$\frac{{G}^{′}}{{G}^{″}} = \frac{1}{tan ( ϕ )} \qquad ( 16 )$$

The phase shift can provide the ratio of the shear storage to the shear loss modulus, e.g., the ratio of the tissue elasticity to the viscosity. Although phase shift by can be quantified using shear or Young's modulus, it can represent a biomechanical parameter independent of changes in the tissue acoustic properties. As the Young's modulus can represent tissue elasticity, the phase shift can be a model-independent biomechanical parameter that can be used to assess the tissue viscoelasticity. Additionally or alternatively, the HMI phase shift can be a localized parameter that can be estimated using the phase of focal displacement and force during the force application. As embodied herein, the relative change in the difference of phase shift degree across the monitoring stage with respect to starting time of treatment (t₀), e.g., Δφ, or the relative change in focal phase shift (Δφ)

Δφ=φ(t)−φ(t₀) (17)

Additionally or alternatively, and as embodied herein, compressive strains can be estimated at adjacent regions of the focal zone in the axial direction (ε_{zz}), which can be estimated through calculation of the spatial derivative of the displacement:

$${ɛ}_{zz} = \frac{\partial {u}_{z}}{\partial z} \qquad ( 18 )$$

where ε can represent the strain tensor, z can indicate the axial direction, and U can represent the displacement vector. As embodied herein, three cases of the axial compressive strains during monitoring for treatment of 10 W at 10, 20, and 30 seconds, respectively, can be performed.

Referring still to FIGS. 15(a)-15(d), FIG. 15(a) is a schematic representing an exemplary HMIFU multi-parametric framework. FIG. 15(b) can represent 1-cycle of HMI focal displacement M-mode. Δφ and displacement can be estimated from values extracted within the focal region. FIG. 15(c) illustrates strain distribution estimated using least square estimation on the same 1-cycle HMI focal displacement M-mode as FIG. 15(b). In FIG. 15(d), Δφ can correspond to the phase angle difference between the registered input force and induced focal displacement. The D in FIG. 15(d) indicates the HMI focal displacement, which can be the peak-to-peak displacement in the oscillatory response under AM-HIFU beam excitation.

### EXAMPLE 2

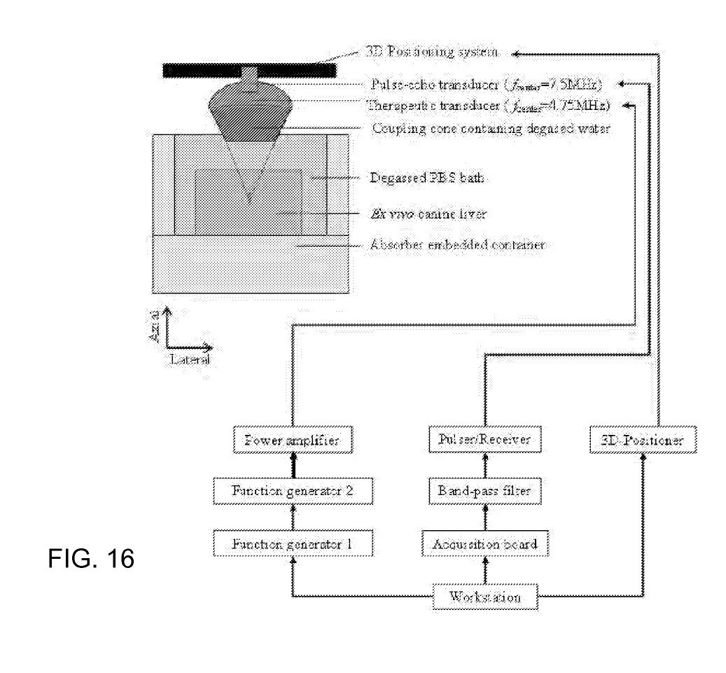

In one example, canine livers (subject=7, lobes=28) were immersed into a degassed Phosphate buffered saline (PBS) solution bath maintained at room temperature. Each specimen was fixed using metallic needles onto an acoustic absorber submerged in a de-ionized and degassed PBS tank. With reference to FIG. 16, an exemplary HMIFU system 100 includes a 4.75 MHz focused Lead Zirconate Titanate (PZT) (e.g., outer diameter 80 mm, inner diameter 16.5 mm, focal depth 9 cm) transducer (Riverside Research Institute, New York, N.Y.) for probing tissue with an AM frequency of 25 Hz, and a confocal 7 .5 MHz single-element pulse-echo transducer (Olympus-NDT, Waltham, Mass., U.S.A.) with a diameter of 15 mm and a focal length of 6 cm for simultaneous RF signals acquisition at a frame rate of 4 kHz. Raster scans were completed by mechanically moving the transducers through a 3D translational system (Velmex Inc., Bloomfield, N.Y., U.S.A.) for targeting and raster scan purposes.

The extrapolated in situ focal acoustic intensity and power was 5546 W/cm², 7164 W/cm², and 9067 W/cm², at 8 W, 10 W, and 11 W, respectively. The treatment power and duration were selected to fall within the boiling regime range and to investigate the performance of HMI under different power and duration used in HIFU. The received RF signals were band-pass filtered (Reactel, Inc., Gaithersburg, Md., U.S.A.) with cutoff frequencies, for example and without limitation, of f_{c1}=5.84 MHz and f_{c2}=8.66 MHz (at −60 dB) and recorded along with the excitation signal representing the force profile and a dual-channel data acquisition unit (Gage applied, Lockport, Ill., U.S.A.) at a sampling frequency of 80 MHz, as shown for example in FIG. 16. Subsequently, a 1-D normalized cross-correlation (e.g., having window size of 3.85 mm and 90% overlap) technique was used to estimate the incremental HMI displacement and axial compressive strains were estimated using a least-square estimator on the RF signals. In each HMIFU treatment, 2D transverse HMI displacement maps were also obtained through raster scan acquisition before and after lesion formation, as described herein. A 3×3 median filter was applied on the displacement profiles in order to enhance the SNR of the displacement map. Lesion-to-displacement contrast values were assessed by taking the ratio of HMI focal displacement outside to that inside of the mapped thermal lesion on the 2D transverse HMI displacement map after the HIFU treatment. Passive Cavitation Detection (PCD) monitoring was also performed, to confirm the presence of tissue boiling at the proposed treatment level, by operating the conically-aligned pulse-echo transducer in passive mode in conjunction with thermocouple measurement. Focal temperature monitoring was performed by inserting a T-type bare wire thermocouple (Omega Inc., Stamford, Conn.), for example having a diameter of 25 μm, inside the tissue. The diameter of the thermocouple was chosen to be smaller than 1/10 of the carrier wavelength to reduce or minimize reflection and viscous heating artifacts.

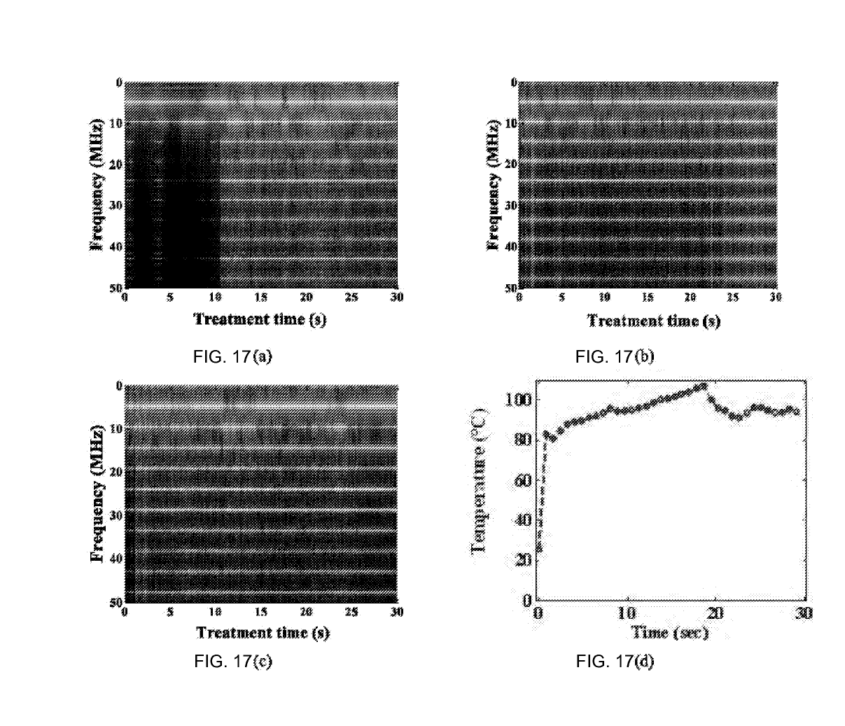

With reference to FIGS. 17(a)-17(d), PCD monitoring was investigated at the three acoustic power levels of 8 W, 10 W, and 11 W for 30 seconds, as illustrated in FIGS. 17(a)-17(c), respectively. As shown for example in FIGS. 17(a)-17(c), each spectrogram detected significant increase in broadband noise, which can indicate formation of strong bubble dynamics induced by tissue boiling.

Referring now to FIG. 17(d), the thermocouple temperature measurement at 8 W can also indicate the presence of boiling. Display of a sharp exponential increment was followed by an unsteady trend around 100° C., which can be due at least in part to the shielding effect due to bubble formation at the focal region. As such, forty-three HIFU lesions were induced across all of the treatment power levels with the presence of boiling throughout this study.

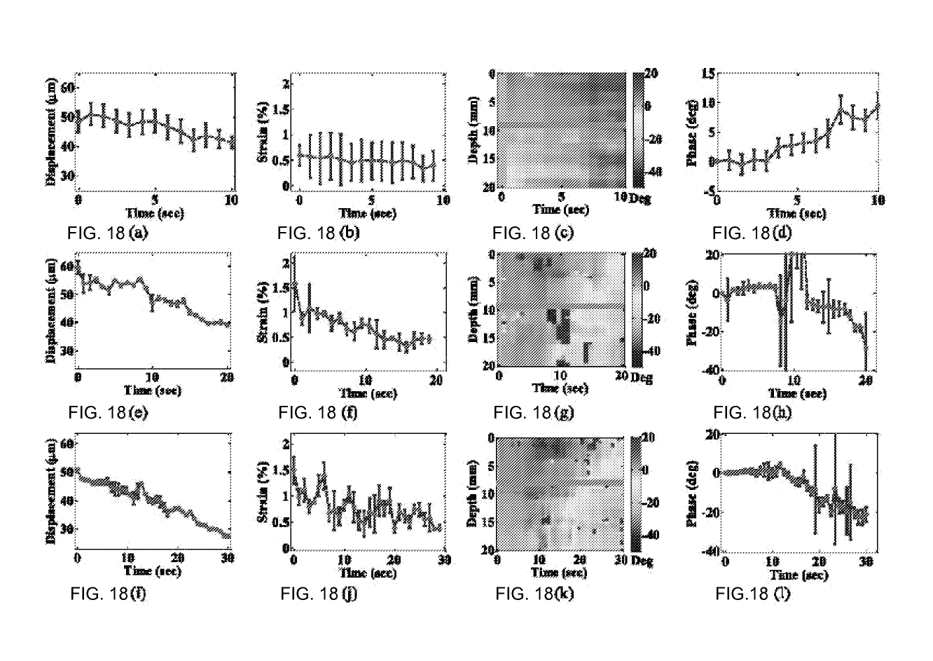

For purpose of illustration and not limitation, nine HMIFU treatments were performed on three samples of ex vivo canine livers. For example and without limitation, HIFU treatments were repeated under acoustic intensity and power of 7164 W/cm² and 10 W for durations of 10-, 20-, and 30-s. With reference to FIGS. 18(a)-18(l), HIFU treatment of acoustic power was set to be 10 W for 10 seconds (FIGS. 18(a)-18(d)), 20 seconds (FIGS. 18(e)-18(h)), and 30 seconds (FIGS. 18(i)-18(l)), respectively. Mean displacement, strain, and phase shift values were estimated across the HIFU focal zone (e.g., approximately 10 mm from the surface of liver as indicated by the thick line on the Δφ M-mode images of FIGS. 18(c), 18(g) and 18(k). As shown, decrease trends were observed for both displacement (FIGS. 18(a), 18(e) and 18(i)) and strain (FIGS. 18(b), 18(f) and 18(j)). Δφ was observed to increase slightly amongst the 10 second cases but decrease significantly amongst the 20 and 30 second cases. The decorrelation points throughout the 2D phase shift M-mode images, as well as phase shifts at the focal zone (FIGS. 18(d), 18(h) and 18(l)), due at least in part to boiling.

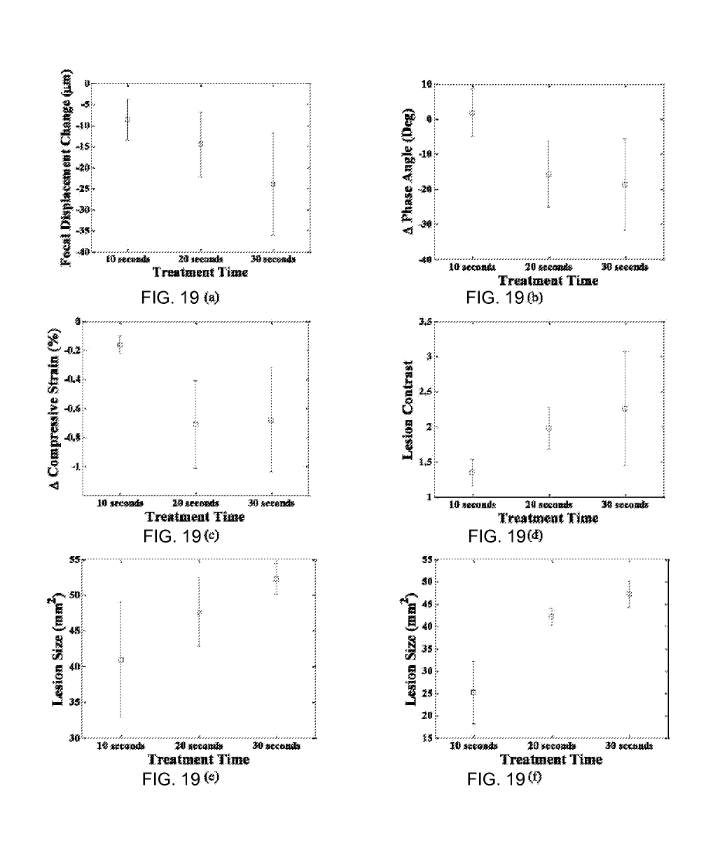

Referring now to FIGS. 19(a)-19(f), statistical summary of the 10 W HIFU treatment was performed. Between three cases of HIFU treatments under 10, 20, and 30 seconds, decrease trend was observed in the peak-to-peak HMI focal displacement value (FIG. 19(a)) and compressive strain (FIG. 19(c)). Δφ (FIG. 19(b)) increased, though unstable, amongst the 10 second treatment cases but illustrated clear decrease trends amongst the 20 and 30 second treatment cases. 2D HMI displacement images observed increase in lesion-to-background displacement contrast (FIG. 19(d)) and lesion size (FIG. 19(e)), which was confirmed with pathology (FIG. 19(f)).

As shown for example in FIGS. 18(a)-19(f), as the treatment duration increased, both the relative change in peak-to-peak value of HMI focal displacement (e.g., −8.67±4.80, −14.44±7.77, −24.03±12.11 μm) and peak axial compressive strain (e.g., −0.16±0.06, −0.71±0.30, −0.68±0.36%) exhibited decrease throughout the treatment. The Δφ showed slight increase at 10 s and significant decrease at 20, 30-s cases (e.g., +1.80±6.80°, −15.80±9.44°, −18.62±13.14°) with a few monitoring time-points around the focal zone where Δφ exhibited an increase in spatial variation.

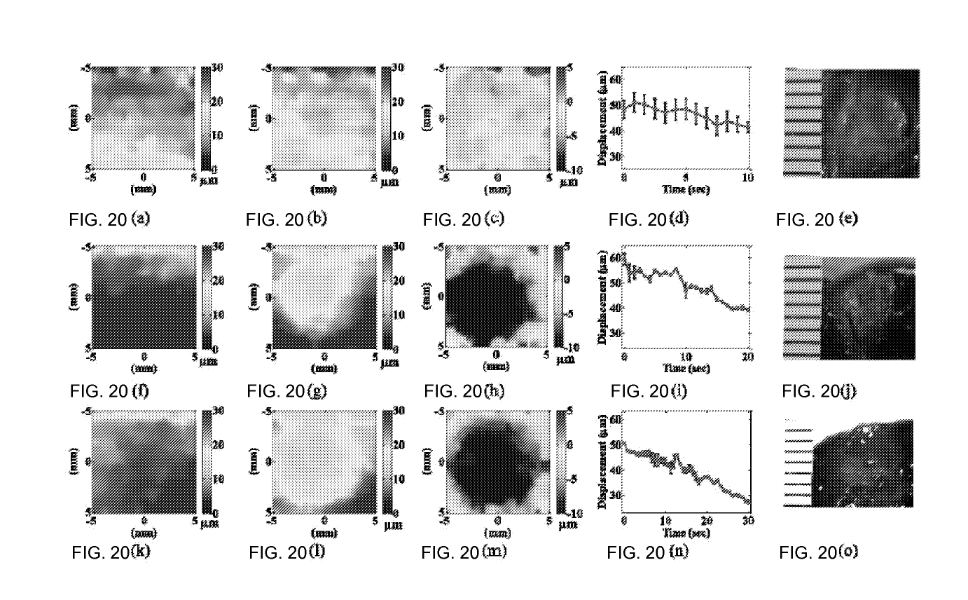

With reference to FIGS. 20(a)-20(o), HMIFU monitoring and assessment images for samples with displacement decrease are shown. HIFU treatment of acoustic power was set to be 10 W for 10 (FIGS. 20(a)-20(e)), 20 (FIGS. 20(f)-20(j)), and 30 seconds (FIGS. 20(k)-20(o)), respectively. Displacement contrast maps (FIGS. 20(c), 20(h) and 20(m)) were estimated from subtracting the displacement maps after lesion formation (FIGS. 20(b), 20(g) and 20(l)) from that of before (FIGS. 20(a), 20(f) and 20(k)) and displayed along with their corresponding monitoring curves (FIGS. 20(d), 20(i) and 20(n)). Both the lesion size and contrast increases with treatment time. The increase in size was confirmed with corresponding gross pathology (FIGS. 20(e), 20(j) and 20 (o)).

Referring now to FIGS. 19(a)-20(o), the standard deviation of both HMI focal displacement and phase shift monitoring curve refers to the average of measurements across the focal zone (2 mm) at the HIFU focusing depth inside the tissue. The 2D HMI displacement images also mapped an increase in lesion-to-background displacement contrast (e.g., 1.34±0.19, 1.98±0.30, 2.26±0.80) and lesion size with treatment time (e.g., 40.95±8.06, 47.6±4.87, 52.23±2.19 mm²), which was verified with pathology results (e.g., 25.17±6.99, 42.17±1.77,47.17±3.10 mm²).

With reference to FIGS. 21(a)-21(o), HMIFU monitoring and assessment images for samples with displacement increase are shown. HIFU treatment of acoustic power were set to be 10 W for 10 (FIGS. 21(a)-21(e)), 20 (FIGS. 21(f)-21(j)), and 30 seconds (FIGS. 21(k)-21(o)), respectively. The displacement contrast maps (FIGS. 21(c), 21(h) and 21(m)) along with their corresponding displacement monitoring curves (FIGS. 21(d), 21(i) and 21(n)) had reversed displacement-to-background contrast in comparison with FIGS. 20(a)-20(o). The mapped lesion sizes increased with treatment time, which was confirmed with pathology images (FIGS. 21(e), 21(j) and 21(o)).

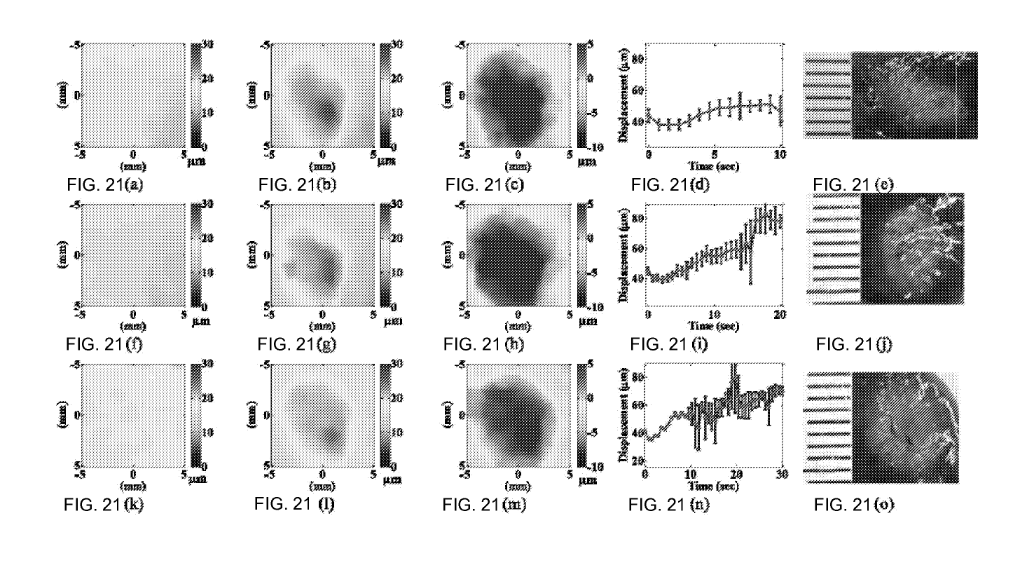

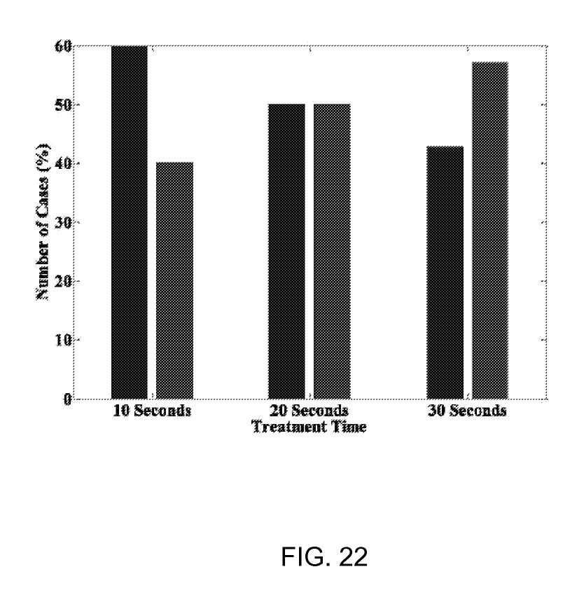

With reference to FIG. 22, a statistical summary change in HMIFU displacement monitoring and contrast map for all treatment samples using 10 W HIFU is illustrated. Each treatment time duration includes samples with both decrease (left bar) and increase (right bar) displacement within the lesion compared to before treatment.

For example and as embodied herein, multi-parametric HMIFU protocol was repeated across three different acoustic powers (8 W, 10 W, and 11 W) under treatment durations of 10-, 20-, and 30-s. With reference to FIGS. 20(a)-20(o), there was a discrepancy in the distribution of the HMI lesion maps including reversed lesion-to-background HMI displacement contrast in the HMI focal displacement decrease lesion samples. By comparison, in FIGS. 21(a)-21(o), where the 2D HMI displacement contrast map (FIGS. 21(c), 21(h) and 21(m)) showed a decrease of displacement inside the thermal lesion HMI focal displacement increase lesion samples, as illustrated in the diagram of FIG. 22.

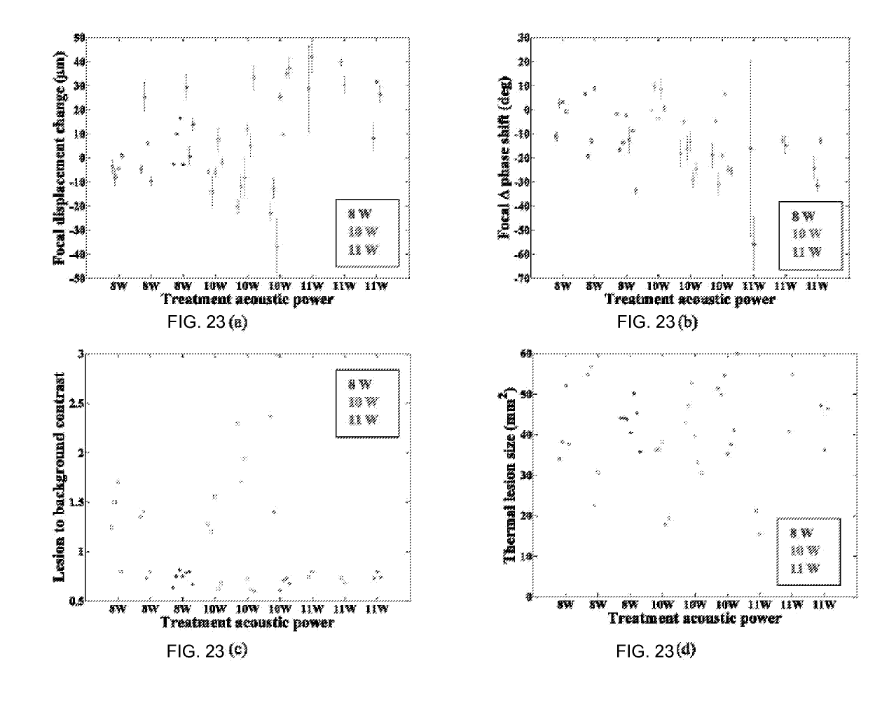

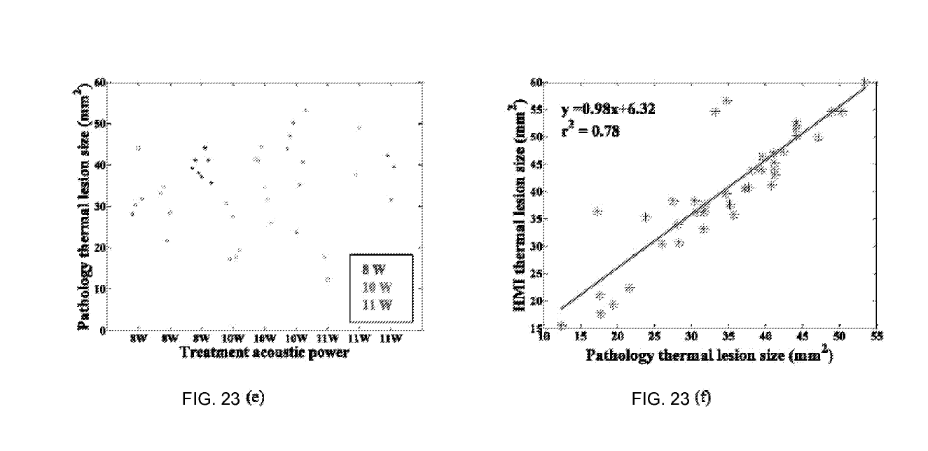

With reference to FIGS. 23(a)-23(f), and Tables 4-6, statistical distribution of the investigated samples at acoustic power of 8 W, 10 W and 11 W for 10 (square), 20 (circle), and 30 (star) seconds, is illustrated. Focal displacement (FIG. 23(a)) increased and decreased across all treatment levels. However, the 10 W sample included most of the decrease cases, and the 11 W samples only included increase cases. Additionally, the magnitude of change increased with treatment time. Trends for Δφ (FIG. 23(b)) illustrated a relatively unstable increase and decrease trend amongst the 10 sec cases, and a decrease trend was observed amongst all other cases across all power levels. Lesion-to-background contrast (FIG. 23(c)) showed a reversed change, among the 8 W and 10 W cases, and 11 W included only increase trends (e.g., contrast<1). Lesion size (FIG. 12(d)) increased with treatment time across all the investigated powers, which was confirmed with gross pathology results (FIG. 23(e)). Linear regression analysis (FIG. 23(f)) was performed for purpose of comparison of the thermal lesion size estimated with gross pathology and HMI mapping. As shown, the HMI mapped size correlated with the pathological findings.

TABLE 4
Quantification of monitoring and assessment parameters across
different acoustic powers for 10 second treatment duration

| | Acoustic Power | | | | |
|---|---|---|---|---|---|
| Parameter | 8 W | | 10 W | | 11 W |
| % of Displacement | 4.9 ± 0.0 | 2.3 ± 2.3 | 13.2 ± 6.0 | 27.3 ± 6.0 | 50.0 ± 23.6 |
| change during | (n = 1) | (n = 4) | (n = 3) | (n = 2) | (n = 2) |
| treatment (%) | | | | | |
| Δ Phase shift during | −2.6 ± 0.0 | −2.9 ± 7.4 | 4.5 ± 6.9 | 1.7 ± 6.1 | −35.8 ± 28.1 |
| treatment (°) | (n = 1) | (n = 4) | (n = 3) | (n = 2) | (n = 2) |
| Lesion-to-background | 1.3 ± 0.0 | 0.7 ± 0.2 | 1.4 ± 0.2 | 0.65 ± 0.04 | 0.77 ± 0.04 |
| contrast after treatment | (n = 1) | (n = 4) | (n = 3) | (n = 3) | (n = 2) |
| Mapped HMI Lesion | 34.0 ± 0.0 | 42.7 ± 8.1 | 36.9 ± 1.1 | 18.6 ± 1.2 | 18.3 ± 4.0 |
| size (mm²) | (n = 1) | (n = 4) | (n = 3) | (n = 3) | (n = 2) |

TABLE 5
Quantification of monitoring and assessment parameters across
different acoustic powers for 20 second treatment duration

| | Acoustic Power | | | | |
|---|---|---|---|---|---|
| Parameter | 8 W | | 10 W | | 11 W |
| % of Displacement | 4.9 ± 0.0 | 2.3 ± 2.3 | 13.2 ± 6.0 | 27.3 ± 6.0 | 50.0 ± 23.6 |
| change during | (n = 1) | (n = 4) | (n = 3) | (n = 2) | (n = 2) |
| treatment (%) | | | | | |
| Δ Phase shift during | −2.6 ± 0.0 | −2.9 ± 7.4 | 4.5 ± 6.9 | 1.7 ± 6.1 | −35.8 ± 28.1 |
| treatment (°) | (n = 1) | (n = 4) | (n = 3) | (n = 2) | (n = 2) |
| Lesion-to-background | 1.3 ± 0.0 | 0.7 ± 0.2 | 1.4 ± 0.2 | 0.65 ± 0.04 | 0.77 ± 0.04 |
| contrast after treatment | (n = 1) | (n = 4) | (n = 3) | (n = 3) | (n = 2) |
| Mapped HMI Lesion | 34.0 ± 0.0 | 42.7 ± 8.1 | 36.9 ± 1.1 | 18.6 ± 1.2 | 18.3 ± 4.0 |
| size (mm²) | (n = 1) | (n = 4) | (n = 3) | (n = 3) | (n = 2) |

TABLE 6
Quantification of monitoring and assessment parameters across
different acoustic powers for 30 second treatment duration

| | Acoustic Power | | | | |
|---|---|---|---|---|---|
| Parameter | 8 W | | 10 W | | 11 W |
| Δ Displacement during | 19.0 ± 0.0 | 73.2 ± 54.1 | 61.4 ± 31.1 | 118.9 ± 61.4 | 48.3 ± 25.9 |
| treatment (μm) | (n = 1) | (n = 5) | (n = 4) | (n = 5) | (n = 3) |
| Δ Phase shift during | −1.9 ± 0 | −14.7 ± 10.6 | −18.5 ± 10.1 | −14.6 ± 18.3 | −22.8 ± 9.4 |
| treatment (°) | (n = 1) | (n = 5) | (n = 4) | (n = 5) | (n = 3) |
| Lesion-to-background | 1.7 ± 0.0 | 0.76 ± 0.05 | 2.3 ± 0.8 | 0.7 ± 0.05 | 0.76 ± 0.04 |
| contrast after treatment | (n = 1) | (n = 5) | (n = 4) | (n = 5) | (n = 3) |
| Mapped HMI Lesion | 44.1 ± 0.0 | 43.3 ± 4.8 | 52.1 ± 2.4 | 43.6 ± 11.2 | 43.4 ± 6.1 |
| size (mm²) | (n = 1) | (n = 5) | (n = 4) | (n = 5) | (n = 3) |

For purpose of illustration and not limitation, HMIFU can be implemented using ultrasound-based elasticity imaging technique, as embodied herein using a pair of confocally-aligned HIFU and pulse-echo transducers for inducing and tracking a stable focal oscillatory motion, which can be related to the local mechanical property. As such, and as embodied herein, HMIFU can perform localized HIFU monitoring without interrupting the treatment. HMIFU can be used to assess tissue relative stiffness, additionally or alternatively, HIFU monitoring utilizing displacement amplitude change. As embodied herein, HMIFU monitoring techniques can be used with high energy HIFU treatment that induced boiling, and can utilize multi-parametric monitoring techniques, including and without limitation, focal displacement, focal compressive axial strain, and relative change in focal phase shift (Δφ). The multi-parametric monitoring techniques described herein can improve the monitoring quality of HMIFU, including and without limitation, under boiling at high energy HIFU treatment, providing complementary analysis with each parameter for indication of various tissue response changes upon formation of a thermal lesion, and/or decoupling of acoustic and mechanical tissue parameters. For purpose of illustration and not limitation, as embodied herein, multi-parametric HMIFU was applied on HIFU treatment monitoring and assessment under three different acoustic powers (8 W, 10 W, and 11 W) and durations (10 s, 20 s, and 30 s).

Additionally, and as embodied herein, across the HIFU treatment cases with boiling, the 2D HMI displacement images underwent reverse lesion-to-background displacement contrast. With reference to FIGS. 20(a)-22, a series of cases where lesions formed followed increased and decreased displacement under same HIFU treatment sequence. For HIFU treatment under 10 W and lasting 10 s (FIGS. 20(a)-20(e) and 21(a)-21(e)), 20 s (FIGS. 20(f)-20(j) and 21(f) and 21(j)), and 30 s (FIGS. 20(k)-20(o) and 21(k)-21(o)), in some cases with focal monitoring displacement decrease during treatment (FIGS. 20(d), 20(i) and 20(n)), and others exhibited an increase trend (FIGS. 21(d), 21(i) and 21(n)). The corresponding displacement contrast images of each treatment also mapped the consistent change of displacement increase (FIGS. 20(c), 20(h) and 20(m)) and decrease (FIGS. 21(c), 21(h) and 21(m)), accordingly. The results of the 10 W treatment cases are illustrated in FIG. 22, and as shown, such results were present across all treatment durations where cases of both increase and decrease displacement were detected. Additionally, as embodied herein, changes in displacement were consistent with its corresponding displacement monitoring curves across all of the treatment cases.

With reference to FIGS. 23(a)-23(f), the outcome distribution of each parameter for each HIFU treatment performed at 8 W, 10 W, and 11 W is illustrated. As shown in FIGS. 23(a) and 23(c), the HMI displacement can increase or decrease after lesioning, and can reflect a change from treatment regardless of the treatment or duration, indicating a consistent effect of boiling. HMI can detect lesions at all treatment durations and powers, and as embodied herein, the HMI displacement contrast ratio can be different from one in every case where a lesion formed. The phase shift (FIG. 23(b)), as embodied herein, can be considered consistent, indicating a decreasing trend, as shown for example in Tables 4-6.

Additionally, and as embodied herein, the mapped lesion sizes from HMI contrast maps (FIG. 23(d)) can be considered consistent with the measurements in the gross pathology findings (FIG. 23(e)). As shown, there can be a correlation between thermal lesion sizes as mapped by HMI and gross pathology findings (FIG. 23(f)). Furthermore, and as embodied herein, with reference to Table 4-6, lesion size can increase from 8 W to 10 W, and can decrease from 10 W to 11 W, due at least in part to a shielding effect associated with attenuation under strong boiling activity. Such reversal in displacement outcomes can occur at least in part due to the measured focal temperature curve and PCD spectrograms, in which the focal region can reach boiling within first few seconds of the treatment window. Beyond a certain temperature threshold, e.g., boiling, in addition or as an alternative to mechanisms such as tissue pulverization, gelatification, or shielding due to bubble occurrence and increase of acoustic absorption can occur with continuous delivery of high thermal dosage. The increase in attenuation can be due at least in part to boiling and/or formation of thermal lesions, and the increase in attenuation can lead to higher displacement at the lesion, which can generate an opposite lesion-to-background contrast. As such, displacement decrease lesion maps can be obtained where the mechanical response change can be considered prevalent (e.g., stiffening occurs) over the structural and/or absorption change, and the displacement increase lesion maps can represent treatments in which the acoustic response change can be considered prevalent (e.g., increase in attenuation). As embodied herein, the average phase shifts exhibited can decrease with heating, including for the 20-and 30-s treatment cases with the 2D HMI maps having either blue or red lesion-to-background displacement ratio, which can indicate a consistent tissue mechanical response change during acoustic property changes. Furthermore, and as embodied herein, the shear modulus of HIFU lesions samples can range between 10 to 15 times compared to that of untreated samples. As such, as embodied herein, lesions can undergo mechanical stiffening, e.g., absorption changes can be prevalent amongst the treatments in which displacement increased.

In addition, and as embodied herein, changes in strain during monitoring can occur, for example on a total number of the treatments performed at 10 W treatment, in which a decrease in the axial compressive strain can occur, which confirmed the ability of HMIFU to confirm relative stiffness monitoring at a finer spatial resolution. Strain estimation can be affected by displacement SNR, as well as the SNR of the displacement profile across the focal zone inside the tissue. As such, the end of the focal excitation zone can be clearly mapped to estimate for the axial compressive strain by calculating the spatial derivative. As embodied herein, the strains can be noisier at other powers. Additionally, and as embodied herein, the Δφ in the 10-sec cases can indicate a slight increase, and in the 20 and 30 second cases can indicate a decrease following an initial increase, as shown for example in FIG. 23(b) and Tables 4-6. As embodied herein, Δφ can decrease amongst all lesions detected, which can indicate Δφ as being an independent biomechanical property marker. Furthermore, and as embodied herein, the 2D HMI displacement images can map the relative increase of the lesion size and lesion-to-background contrast. As shown in Tables 4-6, lesion size and lesion-to-background contrast can decrease from treatments under 20 to 30 seconds, which can result from reduced thermal ablation at the pre-focal shielding effects.

Furthermore, and as embodied herein, treatment assessment can also indicate the capability of HMIFU in displacement mapping across all treatment power levels and durations reaching boiling, demonstrating the effectiveness of HMIFU under high energy HIFU treatment. Decrease in Δφ amongst all of the formed lesions can indicate an independent biomechanical parameter for both monitoring and assessment. HMIFU can thus be robust and reliable in mapping HIFU lesions formed with boiling even with a change in acoustic absorption.

With reference to FIGS. 24(a)-24(i), displacement maps (FIGS. 24(a), 24(d) and 24(g)) and corresponding Δφ maps (FIGS. 24(b), 24(e) and 24(h)) for lesions with decreased displacement under HIFU treatment of 10 W for 10 (FIGS. 24(a)-24(c)), 20(FIGS. 24(d) 24(f)), and 30(FIGS. 24(g)-24(i)) seconds. Referring now to FIGS. 25(a)-25(i), displacement contrast maps FIGS. 25(a), 25(d) and 25(g), and corresponding Δφ maps (FIGS. 25(b), 25(e) and 25(h)) for lesions with increased displacement are shown. The Δφ maps indicate consistent decrease inside the lesion (FIGS. 25(c), 25(f) and 25(i)), which can indicate a consistent biomechanical property change.

As embodied herein, 2D transverse HMI displacement maps can be obtained before and after lesion formation through raster scanning on all treatment cases, and the displacement distribution can be utilized for mapping the lesion size and lesion-to-background displacement contrast. Mapping the lesion using the relative change in phase shift taken from the same displacement and input force acquired at the corresponding raster scan coordinate can be performed. Changes in lesion-to-background displacement contrast across all of the treatment durations are illustrated, for example and without limitation in FIGS. 24(a), 24(d) and 24(g) compared to FIGS. 25(a), 25(d) and 25(g). FIGS. 24(b), 24(e) and 24(h) compared to FIGS. 25(b), 25(e) and 25(h) illustrate a decrease of Δφ within the thermal lesion, which can indicate a change in viscosity-to-elasticity ratio and applicability as an independent biomechanical parameter.

According to another aspect of the disclosed subject matter, multi-parametric HIFU monitoring of HIFU treatment with slow denaturation can be performed. Additionally, for purpose of illustration and comparison, HIFU monitoring of HIFU treatment with slow denaturation can be compared to HIFU monitoring of HIFU treatment with boiling. Techniques for HMIFU can be configured to achieve consistent monitoring of progressive tissue elasticity, which can include initial softening-then-stiffening elasticity phase change due at least in part to the denaturation of protein structures. Such elasticity change can occur, for example and without limitation, during HIFU treatment with lower power and longer duration , and thus can be referred to herein as a “slow” denaturation sequence.

For example, and as embodied herein, monitoring of both acoustic and thermal property change, in addition or as an alternative to mechanical change, can be performed. One or more techniques can be implemented to perform the multi-parametric monitoring, as described herein. Acoustic monitoring can be performed using Passive cavitation detection (PCD) to detect the acoustic response of tissue under HIFU treatment, for example based on characteristics of backscattered HIFU signal spectrum. Broadband noise can be present during tissue boiling, for example with presence of strong bubble dynamics. Additionally or alternatively, focal temperature measurement can be used to provide quantitative information regarding the thermal property change and delivered thermal dosage. As such, HMIFU monitoring under both boiling (high power and short duration) as well as slow denaturation (low power and long duration) HIFU treatment sequence can be performed, based at least in part on the change of HMI displacement and correlation coefficients across the HIFU treatment window.

Additionally, and as embodied herein, thermal and acoustic monitoring can be coupled, for example using thermocouple and PCD monitoring with HMIFU monitoring to determine a suitable power range for consistent monitoring of lesions formed under slow denaturation elasticity change with reduced medium disturbance, such as tissue boiling or acoustic cavitation. The boiling sequence can involve a strong thermal and acoustic change that can affect HMIFU assessment quality, and the slow denaturation sequence can involve a suitable power and duration range that can allow for consistent monitoring of initial stiffening followed by stiffening elasticity change, in addition or as an alternative to consistent phase shift during the lesion formation.

### EXAMPLE 3

In one example, canine livers were immersed into a degassed Phosphate buffered saline (PBS) solution bath maintained at room temperature. Each specimen was fixed using metallic needles onto an acoustic absorber placed inside of a de-ionized and degassed PBS container.

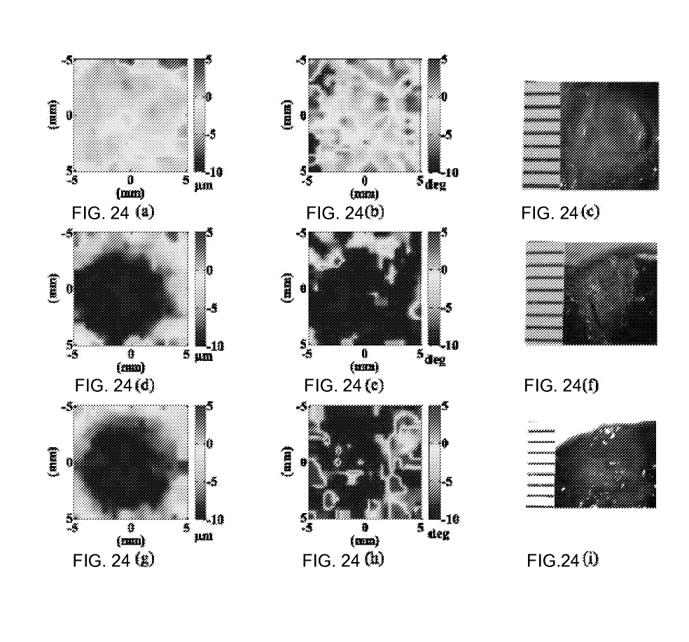

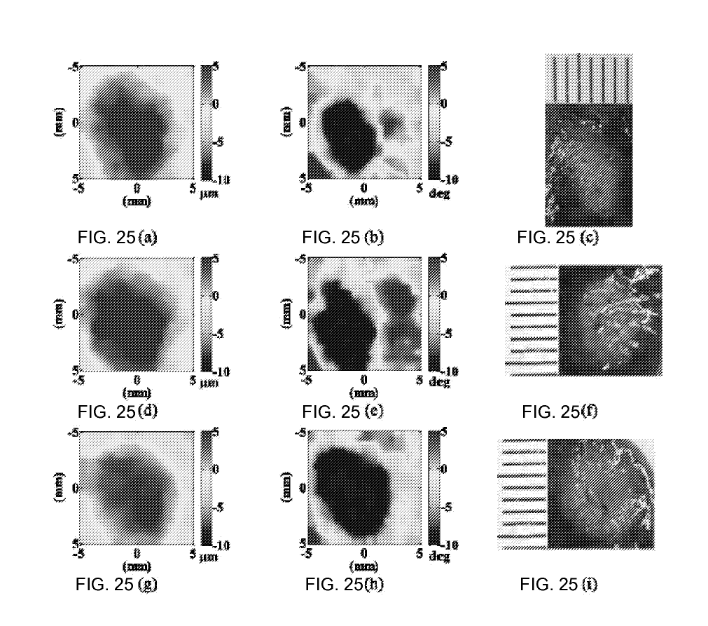

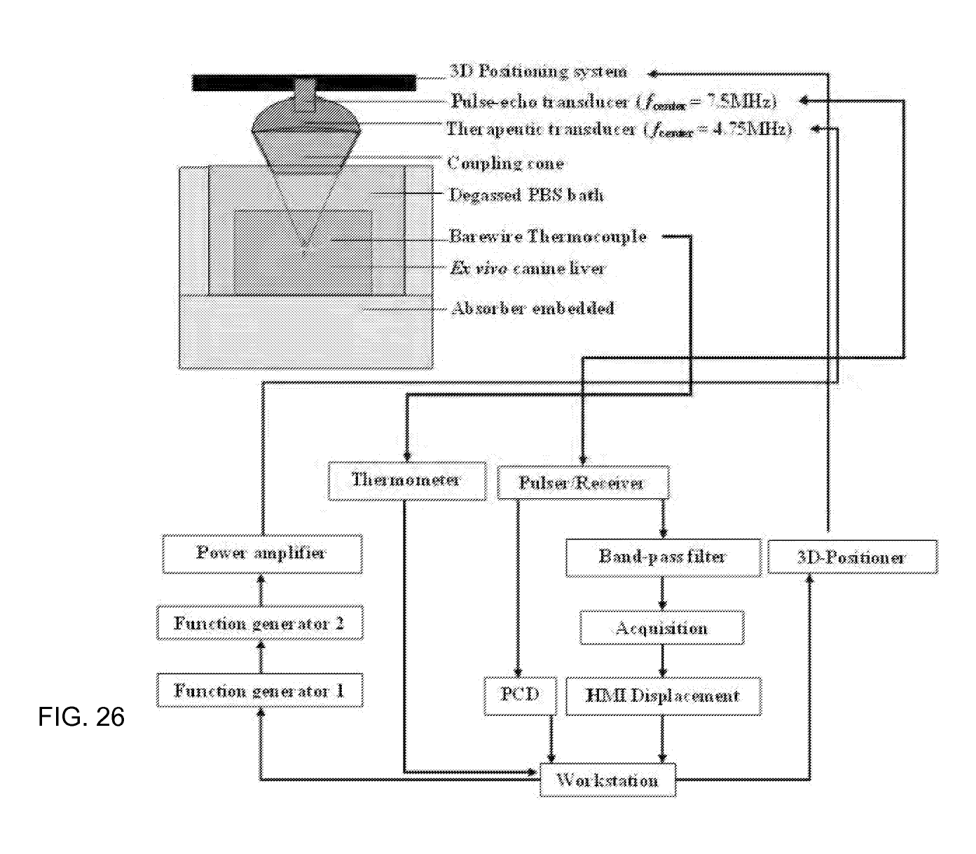

With reference to FIG. 26, an exemplary HMIFU system can include transducers of a 4.75 MHz focused Lead Zirconate Titanate (PZT) (e.g., outer diameter 80 mm, inner diameter 16.5 mm, focal depth 9 cm) transducer (Riverside Research Institute, New York, N.Y.) for probing tissue with an AM frequency of 25 Hz, and a confocal 7.5 MHz single-element pulse-echo transducer (Olympus-NDT, Waltham, Mass., U.S.A.), for example and without limitation, with a diameter of 15 mm and a focal length of 6 cm for simultaneous RF signals acquisition at a frame rate of 1 kHz. As embodied herein, RF signals can be acquired and saved continuously throughout the entire treatment window under the sampling rate of 100 MS/sec, which can provide information about detailed tissue property change throughout the entire HIFU treatment window. The HMIFU system was mounted and controlled through a 3D translational system (Velmex Inc., Bloomfield, N.Y., U.S.A.) for targeting purposes. The received RF signals were band-pass filtered (Reactel, Inc., Gaithersburg, Md., U.S.A.) with cutoff frequencies, for example and without limitation, of f_{c1}=5.84 MHz and f_{c2}=8.66 MHz (at −60 dB) and recorded along with the excitation signal representing the force profile and a dual-channel data acquisition unit (Gage applied, Lockport, Ill., U.S.A.) at a sampling frequency of 80 MHz . A 1-D normalized cross-correlation (for example and without limitation, with window size of 3.85 mm and 90% overlap) technique was used to estimate the incremental HMI displacement and phase shift (Δφ), as described herein.

Additionally, and as embodied herein, peak-to-Peak focal HMI displacements for each treatment case were estimated throughout the treatment window using a peak-detection algorithm. The displacement waveforms were divided into non-overlapping time segments of 1 s. For each segment the local maxima and minima were calculated. The collected local maxima and minima were linearly interpolated, respectively, and the resulting waveforms were smoothed using a moving average filter of 100 points. Constant extrapolation was used to fit and align the resulting waveforms with the displacement waveform. The peak-to-peak amplitude was calculated by subtracting the smoothed local minima waveform from the local maxima waveform. To assess the presence of tissue boiling at the proposed treatment level, PCD monitoring was also performed by operating the conically-aligned pulse-echo transducer in passive mode in conjunction with thermocouple measurement. Focal temperature monitoring was performed by inserting a T-type bare wire thermocouple with diameter of 25 μm (Omega Inc., Stamford, Conn.) inside the tissue through a needle gauge. The diameter of the thermocouple was chosen to be smaller than 1/10 of the carrier wavelength to reduce or minimize reflection and viscous heating artifacts.

Furthermore, and as embodied herein, four additional parameters can be utilized to assess the performance of HMIFU monitoring: Displacement contrast, Mean correlation coefficient, Minimum Correlation coefficient, and PCD Broadband Energy. The displacement contrast c can be represented as:

$$C = \frac{{Disp}_{max} - {Disp}_{min}}{{Disp}_{max} + {Disp}_{min}} \qquad ( 19 )$$

where the Dispₘₐₓ, Dispₘᵢₙ, can represent the maximum and minimum displacement during any monitoring displacement profile during a single treatment window. The mean correlation coefficient pₘₑₐₙ and minimum correlation coefficient pₘᵢₙ can be represented as the average and minimum cross correlation value for the estimated displacement during a single treatment window:

$$\begin{matrix}{ρ}_{mean} = \frac{{\sum }_{i}^{T} ρ ( t )}{T} & ( 20 ) \\ {ρ}_{min} = min ( ρ ( 0 ) \text{:} ρ ( T ) ) & ( 21 )\end{matrix}$$

In addition, and as embodied herein, the PCD Broadband Energy can be obtained by subtracting the harmonic energy and ultra-harmonic energy from the total energy quantified across the PCD spectrograms. As embodied herein, two HMIFU monitoring sequences were performed for HIFU treatment: boiling and slow denaturation, respectively. Monitoring HIFU treatments with slow denaturation were composed of sequences with treatment duration at 120 to 240 seconds under extrapolated in situ focal acoustic intensity and power of 2773 W/cm², 3582 W/cm², 5015 W/cm², at 4 W, 5 W, and 7 W, respectively. Monitoring HIFU treatments with boiling were composed of sequences with treatment duration at 30 seconds under extrapolated in situ focal acoustic intensity and power of 5546 W/cm², 7164 W/cm², and 9067 W/cm², at 8 W, 10 W, and 11 W for treatment with boiling sequence, respectively.

For example, and as embodied herein, 43 HIFU treatment locations were performed on excised canine liver specimens (subject=8, lobes=9) ex vivo. 34 treatments were performed using the slow denaturation treatment sequence, where displacement, cross correlation coefficients, and phase shifts (Δφ) were monitored across the treatment window. In 21 of the treatments (62% of all the treatments), under slow denaturation sequence exhibited displacement decrease following HIFU treatment. Nine treatments were completed using the HIFU treatment with boiling sequence, where six treatments produced displacement increase and 3 treatments produced displacement decrease following HIFU treatment, and no treatments produced an increase-then-decrease progressive displacement change.

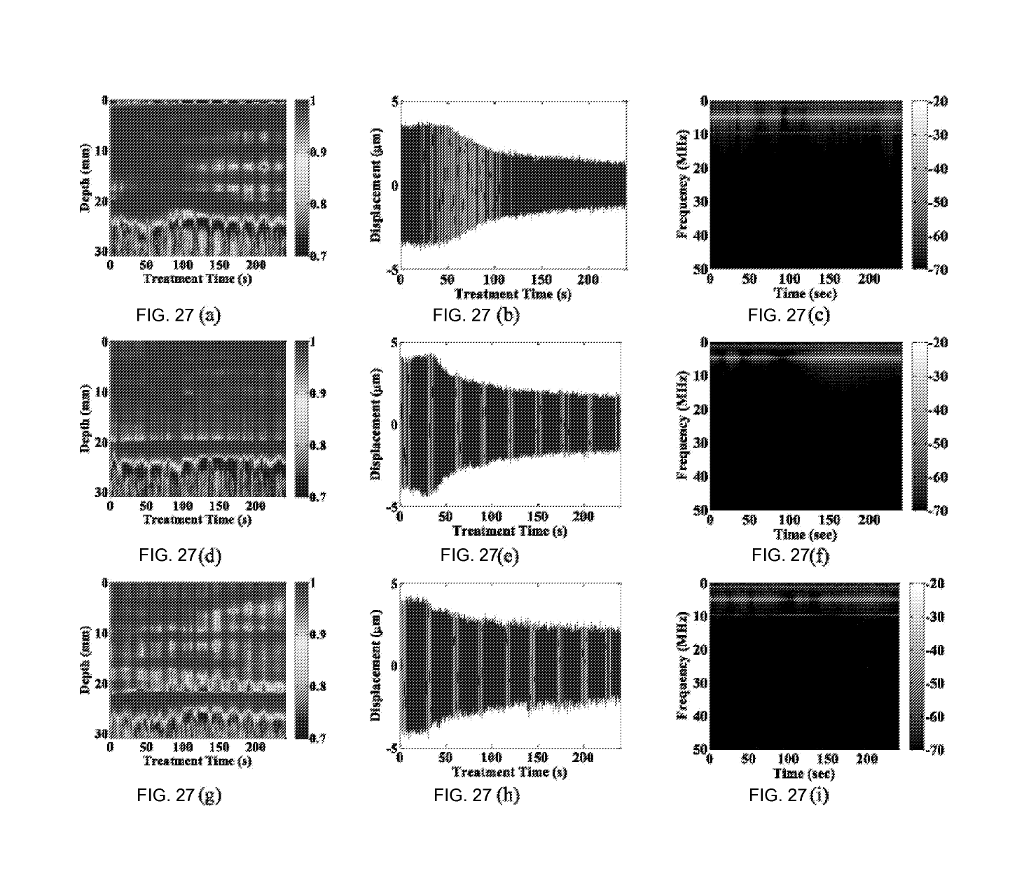

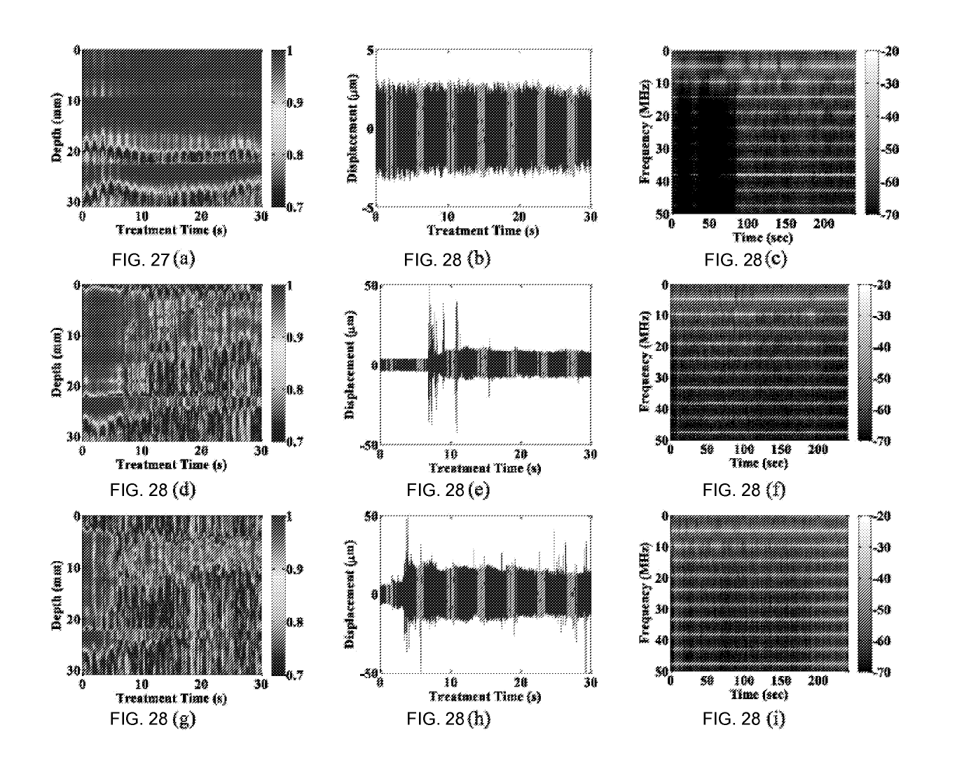

With reference to FIGS. 27(a)-27(i), multi-parametric monitoring of focal HMIFU parameters and acoustic response under HIFU treatment with slow denaturation is illustrated. As shown, the HMIFU correlation coefficient remained high for HIFU treatment with 4 W (FIG. 27(a)), 5 W (FIGS. 27(d)), and 7 W (FIG. 27(g)) as well as focal displacement (FIGS. 27(b), 27(e) and 27(h), respectively). The PCD spectrogram observed little to no presence of bubble dynamics due to boiling, indicated by the presence of broadband level energy (FIGS. 27(c), 27(f) and 27(i), respectively). As illustrated in FIGS. 27(a)-27(i) and Table 7, the averaged displacement change, displacement contrast, phase shift (Δφ), mean correlation, and minimum correlation during the slow denaturation HIFU treatment cases were −36.7±15%, 0.34±0.18, 12.8±2.0°, 0.97±0.079, and 0.81±0.19, which were performed under in situ focal acoustic intensity and power ranged between 2773 W/cm², 3582 W/cm², 5015 W/cm², at 4 W, 5 W, and 7 W, respectively.

TABLE 7
Summary of HMIFU monitoring parameters for slow
denaturation and boiling treatment sequences

| | Monitoring Parameter | | | | |
|---|---|---|---|---|---|
| | Change in HMI | | Mean | Min | |
| | displacement before | ΔPhase shift | correlation | correlation | Displacement |
| Treatment sequence | and after {%} | (°) | Coefficient | Coefficient | contrast |
| Slow denaturation | −36.7 ± 15 | 12.9 ± 2.0 | 0.97 ± 0.08 | 0.81 ± 0.14 | 0.34 ± 0.18 |
| sequences | | | | | |
| Boiling sequence | 197.4 ± 315.3 | 7.0 ± 16.3 | 0.81 ± 0.19 | 0.26 ± 0.4 | 0.46 ± 0.37 |

Referring now to FIGS. 28(a)-28(i), passive cavitation detection (PCD) spectrograms at acoustic power of 8 W (FIGS. 28(a)-28(c)), 10 W (FIGS. 28(d)-28(f)) and 11 W (FIGS. 28(g)-28(i)) all showed increase in broadband noise energy, which can indicate formation of strong bubble dynamics due to boiling. Representative temperature monitoring using T-type bare-wire thermocouple can indicate boiling within the first several seconds from treatment onset, which can indicate presence of unsteady state throughout each treatment monitored.

Additionally, as shown for example in FIGS. 28(a)-28(i) and Table 7, as embodied herein, the averaged displacement change, displacement contrast, phase shift (Δφ), mean correlation, and minimum correlation during the HIFU treatment with boiling cases were 197.4±315.3%, 0.46±0.37, 7.0±16.3°, 0.81±0.14, and 0.26±0.40, which were performed under in situ focal acoustic intensity and power ranged between 5546 W/cm², 7164 W/cm², and 9067 W/cm², at 8 W, 10 W, and 11 W, respectively.

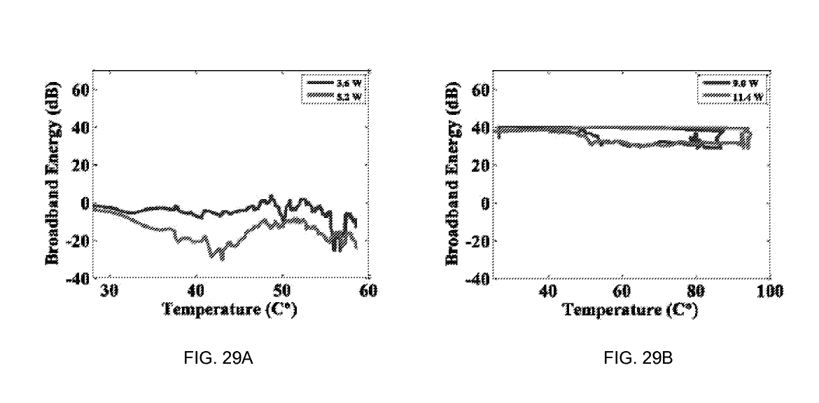

With reference to FIGS. 29(a)-29(b), quantification of broadband energy for PCD monitoring of both HIFU treatment with boiling (FIG. 29(a)) and slow denaturation (FIG. 29(b)) sequences is illustrated. As embodied herein, PCD monitoring was performed on selected cases from each of the treatment sequences. As shown for example in FIGS. 27(c), 27(f), 27(i) and 29(a), no significant presence of broadband noise was detected in slow denaturation HIFU treatment sequences, where HIFU treatment with boiling cases indicated the presence of broadband noise from the backscattered signal on PCD spectrograms with energy level reaching saturation point of the 40 dB detection limit as temperature increases, as shown for example in FIGS. 28(c), 28(f), 28(i) and 29(b).

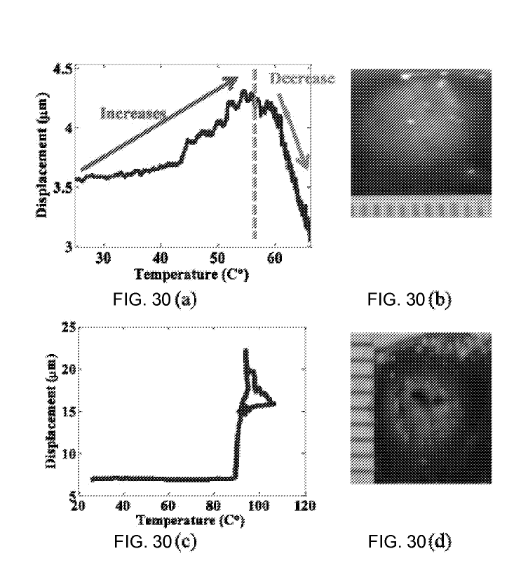

Referring now to FIGS. 30(a)-30g(d), comparative display between the HMIFU focal displacements with temperature monitoring and gross pathology of thermal lesion for both HIFU treatment with slow denaturation (FIGS. 30(a)-30(b), respectively) and boiling (FIGS. 30(c)-30(d), respectively) sequences are shown.

As embodied herein, simultaneous monitoring of PCD and displacement with temperature monitoring were also performed. As shown for example in FIGS. 29(a)-29(b), the energy of broadband noise in PCD spectrograms amongst slow denaturation HIFU treatments showed insignificant presence of broadband noise level ranging between −30 to −40 dB and HIFU treatment with boiling sequences exhibited strong and chaotic trend, reaching saturation of detection limit at 40 dB along with drastic temperature increase up to around 100 degree Celsius. Each of the 21 treatments under slow denaturation HIFU treatment sequence with displacement decrease exhibited an increase-then-decrease displacement trend, that is, the increase slope was 0.072±0.2 μm/sec while the decrease slope was −0.0025±0.03 μm/sec. An example of the slow denaturation displacement curve was shown with simultaneous temperature monitoring using bare-wire thermocouple. With reference to FIGS. 30(a)-30(b), the displacement increase reached a peak range around 50-60 degree Celsius followed by the stiffening phase, and the formed thermal lesion was relatively uniform without significant mechanical tear as observed in the lesions formed by boiling treatment sequences. By comparison with FIGS. 30(c)-30(d), displacement and temperature monitoring were chaotic in the HIFU treatment with boiling, as no consistent pattern between displacement variation and temperature change was indicated.

For purpose of illustration and not limitation, the relationship between HMIFU monitoring parameters (e.g., focal displacement phase shift (Δφ), mean cross correlation coefficients, and minimum cross correlation coefficient) and associated underlying acoustic and thermal property change for both boiling sequence (high power and short duration) as well as slow denaturation (low power and long duration) HIFU treatment sequence is illustrated. For example and as embodied herein, the thermal and acoustic monitoring can be performed using thermocouple and PCD monitoring, respectively. Thermal and acoustic changes can increase the variability of HMIFU assessment during a boiling treatment sequence, and a relatively stable change, e.g., a gradual of tissue softening-then-stiffening indicated by displacement increase then decrease, can occur with the slow denaturation treatment sequence. As such, the presence of acoustic and thermal property changes can be gradual and reduced or minimized.

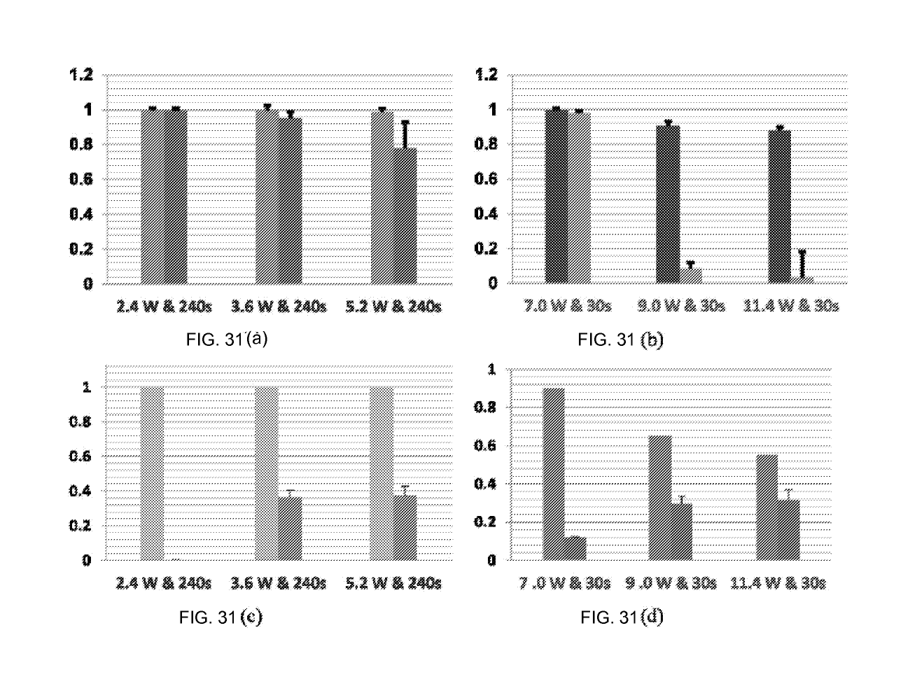

For example, and as embodied herein, for HIFU treatment with slow denaturation, examples of cross correlation (FIG. 27(a), 27(d), 27(g)), focal displacement (FIGS. 27(b), 27(e) and 27(h)) and PCD spectrogram (FIGS. 27(c), 27(f) and 27(i)) were monitored under treatment protocol of 240 seconds with in situ focal acoustic intensity and power ranged between 2773 W/cm², 3582 W/cm², 5015 W/cm², at 4 W, 5 W, and 7 W, respectively. Each of the HMIFU parameters showed consistent trend across the HIFU treatment window: Cross correlation coefficients, including both the mean (as shown for example in FIG. 31(a), left bar) and minimum correlation coefficients (FIG. 31(a), right bar), remained high (as shown for example in FIGS. 27(a), 27(d) and 27(g)) throughout the treatment window.

Additionally, and as embodied herein, focal displacement exhibited increase-then-decrease trend, which can indicate tissue softening-then-stiffening mechanical property change, as shown for example in FIGS. 27(b), 27(e) and 27(h). PCD spectrograms showed reduced or minimized presence of boiling-associated bubble dynamics, as shown for example in FIGS. 27(c), 27(f), 27(i) and 29(a), which can be represented by a reduced or minimized level of broadband noise, as shown for example in FIG. 29(a).

Furthermore, and as embodied herein, for HIFU treatment with boiling, examples of cross correlation (FIGS. 30(a), 30(d) and 30(g)), focal displacement (FIGS. 30(b), 30(e) and 30(h)) and PCD spectrogram (FIGS. 30(c), 30(f) and 30(i)) were monitored under treatment protocol of 30 seconds with in situ focal acoustic intensity and power ranged between 5546 W/cm², 7164 W/cm², and 9067 W/cm², at 8 W, 10 W, and 11 W, respectively. By comparison with the slow denaturation treatments, more significant variations were observed amongst the HMIFU parameters across the HIFU treatment window: The mean cross correlation coefficients remained high, as shown for example in FIG. 31(b), left bar; the minimum correlation coefficients reduced significantly across numerous time points throughout the entire treatment window, as shown for example in FIG. 31(b), right bar, and with reference to FIGS. 28(a), 28(d) and 28(g).

In addition, and as embodied herein, the focal displacement exhibited either relatively unchanged (as shown for example in FIG. 28(b)), or significant increase (FIGS. 28(e) and 28(h)) following HIFU treatment onset. The PCD spectrograms indicated presence of boiling-associated bubble dynamics (FIGS. 28(c), 28(f) and 28(i)), which can be represented by a saturated level of broadband noise across the entire treatment window, as shown for example in FIG. 29(b). With respect to displacement variation and temperature, a gradual displacement increase-then-decrease trend was indicated for HIFU treatment with slow denaturation, and displacement up to around 55° C. following the tissue softening phase occurred, following a decrease trend indicating tissue softening phase, as shown for example in FIG. 30(a). The temperature reading can be affected by the bubble dynamics and shielding effect from the boiling mechanism, and thus a chaotic trend with non-monotonic increase with treatment time can be indicated, as shown for example with FIG. 30(b).

Additionally, and as embodied herein, characteristic differences between the gross pathological lesion formed after the two different HIFU treatment sequences can be determined. For example, and as embodied herein, lesions formed under slow denaturation sequence can be relatively uniform in shape and boundary as shown for example in FIG. 30(c). Lesion formed under HIFU treatment with boiling can include cavities, which can be due at least in part to strong mechanical and/or thermal effects from the boiling mechanisms around 100 degree Celsius, as shown for example in FIG. 30(d). For example, and as embodied herein, mean correlation coefficients remained high throughout the treatment duration for both HIFU treatment with slow denaturation and HIFU treatment with boiling, as shown for example in FIGS. 30(a)-30(b). The minimum correlation reduced in average down to 0.26 amongst the HIFU treatments with boiling (as shown for example in FIG. 30(b)) compared to the slow denaturation cases, which remained above 0.8 in average (as shown for example in FIG. 30(a)). As an increase-then-decrease displacement trend was observed for all of the slow denaturation with displacement decrease (as shown for example in FIG. 31(c)), more of the HIFU treatments with boiling exhibited a trend of displacement increase or no change (as shown for example in FIG. 31(d)). A strong displacement contrast occurred across the monitoring displacements for all of the HIFU treatments with boiling, as shown for example in FIG. 31(d), which can indicate effectiveness of HMI displacement even under the presence of strong broadband noise induced by the boiling bubble dynamics.

Furthermore, and as embodied herein, slow denaturation sequences can be suitable to monitor a steady viscoelasticity change under HIFU treatment using HMIFU. Such techniques can maintain a high cross correlation coefficient while reducing or minimizing disturbance due to the mechanical and acoustical noise induced by boiling mechanism, including at temperatures at 100° C. or more. Additional factors that can affect the level and occurrence chance of boiling mechanism can include, for purpose of illustration and not limitation, degassing time, depth dependent attenuation effect. Displacement contrast can remain in treatments with strong broadband energy level and low minimum correlation coefficient, and thus the effectiveness of HMIFU to detect the formation of lesion even under HIFU treatment with boiling can be confirmed.

The foregoing merely illustrates the principles of the disclosed subject matter. Various modifications and alterations to the described embodiments will be apparent to those skilled in the art in view of the teachings herein. It will thus be appreciated that those skilled in the art will be able to devise numerous techniques which, although not explicitly described herein, embody the principles of the disclosed subject matter and are thus within its spirit and scope.

1. A computer-implemented method for estimating acoustic attenuation in a tissue from time-varying radiation force information generated through the application of acoustic energy to the tissue from at least first and second focal depths, comprising:
acquiring first signals representing oscillatory motion of the tissue in response to the radiation force proximate the first focal depth;
acquiring second signals representing oscillatory motion of the tissue in response to the radiation force proximate the second focal depth;
estimating, by the processor, the oscillatory motion of the tissue from each of the first and second signals; and
estimating, by the processor, the acoustic attenuation in the tissue from the estimated oscillatory motion of the tissue from the first and second signals.

2. The method of claim 1, further comprising applying the acoustic energy by pulsing a focused ultrasound transducer at a modulation frequency.

3. The method of claim 1, wherein acquiring each of the first and signals comprises pulsing an imaging transducer configured as a pulser/receiver to acquire radio frequency signals at a pulse repetition frequency.

4. The method of claim 3, wherein estimating the oscillatory motion of the tissue from each of the first and second signals comprises applying 1D normalized cross correlation to the acquired radio frequency signals.

5. The method of claim 1, wherein estimating the acoustic attenuation comprises linearly correlating the estimated oscillatory motion from each of the first and second signals.

6. The method of claim I, further comprising estimating the acoustic attenuation at a first portion of the tissue, estimating the acoustic attenuation at a second portion of the tissue lateral from the first portion, and determining a displacement map of the tissue using the estimated acoustic attenuation of the first portion and the estimated acoustic attenuation of the second portion.

7. A system for estimating acoustic attenuation in a tissue, comprising:
an ultrasound transducer configured to apply acoustic energy to the tissue a first focal depth and a second focal depth to generate a time-varying radiation force proximate the first focal depth and the second focal depth;
an imaging transducer configured to be optically coupled to the tissue and acquire first signals representing oscillatory motion of the tissue in response to the radiation force proximate the first focal depth and second signals representing oscillatory motion of the tissue in response to the radiation force proximate the second focal depth;
one or more memories; and
one or more processors coupled to the one or more memories and the imaging transducer, wherein the one or more processors are configured to:
estimate the oscillatory motion of the tissue from each of the first and second signals; and
estimate the acoustic attenuation in the tissue from the estimated oscillatory motion of the tissue from the first and second signals.

8. The system of claim 7, wherein the one or more processors is coupled to the ultrasound transducer and further configured to pulse the ultrasound transducer at a modulation frequency.

9. The system of claim 7, wherein the imaging transducer is configured as a pulser/receiver, and the one or more processors is further configured to pulse the imaging transducer at a pulse repetition frequency to acquire radio frequency signals corresponding to each of the first and second signals.

10. The system of claim 9, wherein the one or more processors is further configured to estimate the oscillatory motion of the tissue from each of the first and second signals by applying ID normalized cross correlation to the acquired radio frequency signals.

11. The system of claim 7, wherein estimating the acoustic attenuation comprises linearly correlating the estimated oscillatory motion from each of the first and second signals.

12. The system of claim 7, further comprising a positioning apparatus coupled to the ultrasound transducer and logically coupled to the one or more processors, the positioning apparatus configured to move the ultrasound transducer to aim the ultrasound transducer at the first focal depth and the second focal depth in response to the one or more processors.

13. The system of claim 12, wherein the positioning apparatus is further configured to aim the ultrasound transducer to a first portion of the tissue and a second portion of the tissue lateral from the first portion in response to the one or more processors, the one or more processors further configured to:
estimate the acoustic attenuation at each of the first portion and second portion of the tissue, and
determine a displacement map of the tissue using the estimated acoustic attenuation of the first portion and the estimated acoustic attenuation of the second portion.

14. The system of claim 7, wherein the imaging transducer is coupled to and coaxially aligned with the ultrasound transducer.
